(19) United States
(12) Patent Application Publication
(10) Pub. No.: US 2006/0130565 A1
(43) Pub. Date: Jun. 22, 2006

(54) Measurement of total reactive isocyanate groups in samples using bifunctional nucleophiles such as 1,8-diaminonaphthalene (dan)

(76) Inventors: Robert P Streicher, Cincinnati, OH (US); Dhimiter Bello, Belmont, MA (US); Susan R Woskie, Cambridge, MA (US); Ardith A Grote, Fairfield, OH (US)

Correspondence Address: NEEDLE & ROSENBERG, P.C., SUITE 1000, 999 PEACHTREE STREET, ATLANTA, GA, 30309-3915, US

(21) Appl. No.: 10/536,219

(22) PCT Filed: Dec. 1, 2003
(86) PCT No.: PCT/US03/38051

§ 371 Date: Sep. 23, 2005

Related U.S. Application Data
(60) Provisional application No. 60/429,963, filed on Nov. 29, 2002.

(51) Int. Cl. G01N 33/00; C08G 18/00

(52) U.S. Cl. 73/61.41; 73/23.2

(57) ABSTRACT

The present invention provides for a method for detecting the presence of isocyanate in a sample by (a) contacting an isocyanate derivatizing agent with a sample under conditions suitable to the formation of a reaction product capable of detection and (b) detecting the presence or absence of the reaction product as an indication of the presence or absence of isocyanate in said sample. The present invention also provides a method of quantifying the total isocyanate presence by quantifying the reaction product An organic compound useful for detecting the total quantity of isocyanate in an environmental sample is disclosed. The compound is 1,8-diaminonaphthalene (DAN), a bifunctional nucleophilic derivatizing agent. Methods for detecting a particular isocyanate monomer or the total isocyanate in environmental samples using DAN and related isocyanate derivatizing agents are provided.

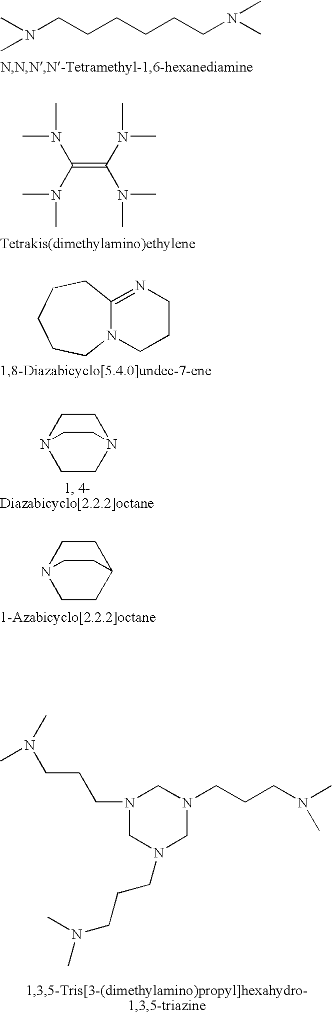

### CROSS REFERENCE TO RELATED APPLICATIONS

This application claims the benefit of U.S. Provisional Application Ser. No. 60/429,963, filed Nov. 29, 2002, herein incorporated by reference in its entirety.

### ACKNOWLEDGEMENTS

The invention was made by at least one inventor in the Centers for Disease Control and Prevention National Institute for Occupational Safety and Health (NIOSH), an agency of the U.S. government. The U.S. government has certain rights in the invention.

### BACKGROUND OF THE INVENTION

1. Field of the Invention

This invention relates generally to the fields of organic and analytical chemistry and, more particularly, to compounds and methods for the detection and/or measurement of isocyanates.

2. Background

Isocyanates are a class of chemicals, organic compounds containing the isocyanate functional group —N═C═O, used in the production of a wide variety of chemical compounds. These chemical compounds are in turn incorporated into a vast number of products which are used in great quantities world-wide.

Monoisocyanates are used as intermediates in the production of herbicides, crop protection agents, and anti-diabetic pharmaceuticals, while long-chain aliphatic monoisocyanates are used for the surface treatment of textiles. Diisocyanates and polyisocyanates are intermediates in the manufacture of polyurethane materials. These materials include rigid foams for insulation, flexible foams for seating, and paints yielding durable finishes. In preparing these and other commercial products, manufacturers utilize the various species of isocyanates, both alone and in combination, in order to obtain the desired characteristics in the final product. The most common isocyanates employed in industry are 2,4- and 2,6-toluene diisocyanate (TDI), 4,4′-diphenylmethane diisocyanate (MDI), hexamethylene diisocyanate (HDI), and isophorone diisocyanate (IPDI).

While isocyanates provide a great many commercial benefits, their use is, unfortunately, accompanied by certain problems; they have been known to create significant health risks. One of the most serious of these is the effect of isocyanates on the human respiratory system. They can also cause irritation of the eyes and other mucous membranes.

Upon inhalation, isocyanates act as respiratory irritants. Fortunately, in the short term, the symptoms resulting from isocyanate inhalation usually disappear after removal of the person from the contaminated environment. Repeated exposure to isocyanates over a prolonged period, however, can lead to progressive and permanent impairment of pulmonary function. This impairment manifests itself in the form of shortness of breath and increased stress on the heart. More seriously, a “sensitized” condition arises in approximately 5% of all persons exposed to isocyanates. In this condition, asthmatic symptoms present themselves almost immediately upon exposure to even relatively low concentrations of isocyanates, i.e., concentrations which do not affect those who are not sensitized to isocyanates. Exposure to isocyanate compounds is a major cause of occupational asthma among workers.

During the 1990s, animal tests showed that dermal exposure could give rise to respiratory sensitization. Air sampling methods for isocyanates were then adapted for sampling surfaces, but the increased level of contamination of a surface sample compared to air raised the limit of detection and made results difficult to interpret.

Because of the serious adverse health risks associated with the use and inhalation of isocyanates by industrial workers, most industrialized countries have set limits on the permissible levels of exposure. For example, the National Institute for Occupational Safety and Health (NIOSH) in the United States has set a level of 5 ppb; the Deutsche Forschungsgemeinschaft in the Federal Republic of Germany has a set limit of 10 ppb; and the Health and Safety Executive of the United Kingdom has created a standard of 20 μg NCO m⁻³ for an eight hour time-weighted average and 70 μg NCO m⁻³ for a ten minute time-weighted average.

Attention has been given to devising various methods for the detection of isocyanates and the measurement of exposure levels. Isocyanate exposure typically is to a complex mixture of these compounds. Established methods of measuring exposure have important limitations. Some methods can measure only compounds for which analytical standards are available; some are not quantitative because they give different responses for different isocyanates or are incapable of detecting some isocyanate species. Some do not accurately measure isocyanate present as aerosol. Some methods are not very sensitive.

Several of these established methods are concerned with determining the concentration of particular airborne isocyanate species in an environment, while others are directed toward measuring the residual isocyanate monomer content in various isocyanate starting materials, e.g., bulk isocyanate prepolymers and the like. In addition, methods for determining the percentage of free isocyanate groups present in urethane-based polymers have also been developed. Although such methods are predominantly spectrophotometric or chromatography-based, polarography, potentiometry, dielectric constant determination, detector tubes, impregnated paper tapes, and coated piezoelectric crystals have also been used. An overview of various methods presently available for the detection of isocyanate species in air is provided in Dharmarajan et al., “Recent Developments in the Sampling and Analysis of Isocyanates in Air, Sampling and Calibration for Atmospheric Instruments,” Am. Soc'y for Testing & Materials, Spec. Tech. Pub. No. 957, pp. 190-202 (1987), and Purnell et al., “Methods for the Determination of Atmospheric Organic Isocyanates: A Review,” Analyst, 110, 893-905 (1985).

In many environments, the hazard posed by isocyanate contamination is not limited to a single isocyanate species. Products made using isocyanates may contain several different isocyanate species, and new species may be released during the use of the product. Therefore, it is important to assess the total hazard resulting from exposure to isocyanates, which requires measuring all isocyanate species. While the detection and quantification of particular isocyanate species are important, detection and quantification of the total number of isocyanate groups present in an environment, regardless of the species which are present, can be just as important. This arises from the fact that the health risks mentioned previously may not occur only as a result of exposure to a single isocyanate species. In recognition of this, the United Kingdom has adopted an exposure standard for isocyanates which is based upon the total isocyanate groups present in an environment. Silk et al., Ann. Occup. Hyg., 27 (4), 333-39 (1983). However, analytical standards are unavailable for a majority of these species, preventing individual identification of all isocyanate species in routine sample analysis. Almost all of the methods reviewed by Purnell, et al. (1985) and Dharmarajan, et al. (1987) are based upon the measurement of certain individual isocyanate species, and therefore, cannot measure total isocyanate concentration.

One particular established method which could conceivably be used to determine the total isocyanate groups present in a sample is described in Marcali, “Microdetermination of Toluenediisocyanates in Atmosphere,” Anal. Chem., 29 (4), 552-558 (1957). The method described in that article (originally intended by its developers to enable the detection of trace quantities (down to 10-20 ppb) of toluenediisocyanates in air) comprises initially hydrolyzing a toluenediisocyanate (TDI) monomer to prepare a derivative thereof, i.e., a toluenediamine (TDA).

Diazotization of the TDA, and subsequent coupling of the stable diazo compound with N-1-naphthylethylenediamine, is then undertaken. This results in the production of a compound having a reddish-blue color, which compound may be measured spectrophotometrically to determine the isocyanate level.

The Marcali method is limited to the measurement of aromatic isocyanates. Furthermore, the Marcali method is susceptible to interferences, exhibits poor sensitivity (in the range of 20 ppb) when compared with standard chromatographic methods, and the response varies with isocyanate structure. For example, it was found that TDA and any other aromatic amines present in the sample were also diazotized and bonded to the N-1-naphthylethylene diamine. TDA present in the air sample results in a false positive reading for TDI. Additionally, quantification of isocyanates can only be accomplished for those species for which the response factor is already known. Mixtures of isocyanates for which the response factors are known also cannot be accurately quantified without knowing their relative amounts. For example, 2,4-TDI and 2,6-TDI, which are typically found together in products, have different response factors. As such, obtaining an accurate measure of the total isocyanate species depends upon knowing the relative amount of each.

Another method currently used to measure isocyanates is Method 25 for the Determination of Hazardous Substances (MDHS 25) of the Health Safety Executive of the United Kingdom. The method, which has been used for detecting the total isocyanate presence in a sample, involves derivatizing isocyanates by forming ureas therefrom using 1-(2-methoxyphenyl)piperazine (MOPP): 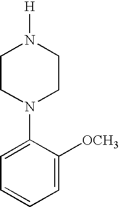
The resulting ureas are analyzed using high performance liquid chromatography (HPLC) equipped with ultraviolet (UV) and electrochemical (EC) detectors in series. Isocyanate-derived peaks are identified on the basis of their UV/EC response ratio, and all such peaks are quantified using an isocyanate monomer standard. The total airborne isocyanate concentration is calculated from the sum of all isocyanate-derived HPLC peaks as described in “Health and Safety Executive: MDHS 25, Methods for the Determination of Hazardous Substances: Organic Isocyanates in Air,” Health & Safety Executive/Occupational Safety and Hygiene Laboratory (March 1987).

Bagon, et al. (Am. Ind. Hyg. Assoc. J., 45(1): 39-43, 1984) disclose the use of MDHS 25 for determining isocyanate monomers and prepolymer relative to a monomer standard. The MDHS 25 method has been found to be unreliable in its ability to correctly identify isocyanate species and inaccurate in its quantitation of those species (Streicher, et al., “Investigation of the Ability of MDHS 25 to Determine Urethane-Bound Isocyanate Groups,” Am. Ind. Hyg. Assoc. J., 56: 437-42, 1995). An evaluation of this method revealed that neither detector (UV or EC) response was found to be proportional to the number of derivatized isocyanate groups present in model urethane oligomers. See Streicher et al., 1995. This non-proportional response makes the method unreliable in terms of both its ability to correctly identify isocyanate species and inaccurate in its quantification of such species. Moreover, it would be advantageous if a means were available whereby certain properties, e.g., detectability, could be improved over that provided by MOPP.

Schmidtke, et al. (Fresenius J. Anal. Chem., 336(8): 647-54, 1990) teach a sensitive high performance liquid chromatographic procedure to analyze hexamethylene diisocyanate (HDI), 2,4- and 2,6-toluene diisocyanate (TDI), and 4,4′-diphenylmethane diisocyante (MDI) in air. The isocyanates are trapped on a sorbent coated with 1-(2-methoxyphenyl)piperazine (MOPP). The resulting derivatives are separated using a column switching technique employing either a diode array UV detector or an electrochemical detector.

Another reaction scheme which has been considered in an effort to quantify the total isocyanates in a sample, e.g., air, involves passing the air through an impinger containing propanol under favorable conditions, wherein the isocyanate species react with propanol to yield their respective propyl carbamates. The excess propanol is then removed from the reacted mixture, the carbamate is subsequently hydrolyzed, and the resulting propanol is analyzed. The amount of propanol provides a quantification of the total isocyanates present. See Robertson, “Determination of Total Isocyanate Concentrations In Air By Headspace Gas Chromatography,” Section Paper of the Health & Safety Executive, Research & Laboratory Services Division (1986).

This methodology, however, suffers from at least three drawbacks. First, since the derivatizing reagent and the analyte are identical (propanol), the derivatizing reagent may introduce inaccuracies into the analysis if it is not completely removed after the formation of the carbamate. Secondly, the conditions required to regenerate the propanol from the carbamate are relatively harsh. Thirdly, the rate of the hydrolysis reaction varies substantially with the various isocyanate species.

Yet another method utilizes tryptamine: 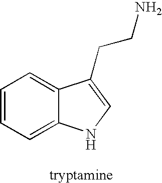
as the derivatizing reagent for isocyanate. After derivatization, HPLC with fluorescence and electrochemical detection in series is employed. See Wu et al., “Application of Tryptamine as a Derivatizing Reagent for the Determination of Airborne Isocyanates: Part 3. Evaluation of Total Isocyanates Analysis by High-Performance Liquid Chromatography with Fluorescence and Amperometric Detection,” Analyst, 115, 801-807 (1990); Wu, et al., Analyst, 116(1): 21-25 (1991).

The Wu et al. method is similar to MDHS 25. Although the Wu et al. method appears to give more selective detection with less response factor variability than the MDHS 25 method, all compounds must elute as observable peaks, and the analysis assumes that all isocyanates derived from a particular monomer have the same detector response factor. However, it has been found that the detector response factors of several tryptamine-derivatized isocyanates vary significantly. This method also requires the use of two detectors to confirm the identity of peaks as derivatized isocyanates.

While this method provides a relatively higher degree of selective detection and a less-variable response factor than the method which uses 1-(2-methoxyphenyl)piperazine, there are certain aspects that could be improved upon.

For example, a reagent which reacts with isocyanates faster than tryptamine would be desirable. This is because the more reactive the reagent is with an isocyanate, the smaller is the problem of losses of isocyanates to side reactions after the isocyanate is collected but prior to derivatization. Secondly, a reagent that provides a greater detector response than tryptamine would enable determination of the quantity of isocyanates at lower concentration levels. Finally, a reagent that yields derivatized isocyanates whose detector responses vary less than those derived from tryptamine would enable a more accurate identification and quantification of isocyanate species.

Wu et al. (Am. Ind. Hyg. Assoc. J. 47(8): 482-87, 1986) describe a procedure for detecting isophorone diisocyanate (1-isocyanato-3-isocyanatomethyl-3,5,5-trimethylcyclohexane) by drawing air through a solution containing one of the following derivatizing agents: 1-(2-methoxyphenyl)piperazine, N-(4-nitrobenzyl)propylamine, or dibenzylamine. The reaction forms the corresponding urea derivatives which are then determined by HPLC using a LiChrosorb® RP-18 column.

Other reagents have also been utilized to derivatize isocyanates, rendering them analyzable. For example, 9-(methylaminomethyl)anthracene (MAMA): 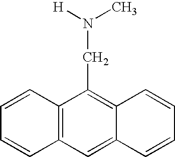
and 1-(2-pyridyl)piperazine: 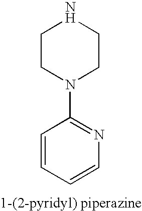
have been used in an effort to determine the presence of particular isocyanate species present in a sample. MAMA has recently been used also to determine total isocyanate.

Hanus, et al. (Mikrochimica Acta, 3(⅙): 197-206, 1988) disclose the use of tubes packed with Chromosorb® WAW, end-plugged with glass wool and impregnated throughout with 1-(2-pyridyl)piperazine for collection and in situ derivatization of toluene 2,4-diisocyanate (TDI), 4,4′-diphenylmethane diisocyanate (MDI), and 1,6-hexamethylene diisocyanate (HDI), which are collected from air.

The compounds are desorbed and detected by ion-pair chromatography using a LiChrosorb® RP-18 column.

U.S. Pat. No. 3,533,750 to Belisle discloses a process for detecting toluene diisocyanate, other aromatic isocyanates, or aromatic amines in ambient air. The method involves contacting an air sample with an acid solution of glutaconic aldehyde and then with a cationic ion exchange resin. The isocyanate is converted to a corresponding amine that is reacted with a reagent to produce a yellow color that is concentrated on the surface of the resin. Although the method is quick and sensitive, it cannot be used to detect aliphatic isocyanate species.

Dalene, et al. (J. Chromat., 435: 469-81, 1988) disclose a high performance liquid chromatographic method for the trace analysis of complex air mixtures containing 2,6- and 2,4-toluene diisocyanates and related amino isocyanates and diamines. The method is based on derivatization of the isocyanate functional groups to corresponding urethane groups with alkaline ethanol as the sampling and reacting medium.

The determination of isocyanates in air by normal-phase liquid chromatography with fluorescence detection is described by Kormos, et al. (Anal. Chem. 53(7): 1122-25, 1981). The isocyanates are converted to the N-methyl-1-naphthalenemethylamine (MNMA) urea derivatives.

The foregoing methods are incapable of correctly identifying or accurately quantifying all isocyanate species that may be present in a sample. Thus, there is a need for a simple method for detecting total isocyanate in an environmental sample, such as a solid, liquid, or air sample, or a surface wipe sample.

Thus, there exists a need for a method which will provide a relatively safe and simple means for the quantitative detection of all isocyanate species present in a sample especially at low concentrations. Moreover, there exists a need for a method which minimizes the problems associated with the detection of isocyanates due to their instability.

U.S. Pat. No. 5,354,689 by Streicher issued Oct. 11, 1994 entitled “Method of Detecting Isocyanates” discloses a method for detecting the presence of isocyanate in a sample comprising (a) contacting an isocyanate derivatizing reagent having the formula
R—R′,
wherein R is 9-anthracenylmethyl or a derivative thereof and R′ is a radical having a single isocyanate-derivatizing functionality comprising a cyclic secondary amine with a sample under conditions suitable for the formation of a reaction product capable of detection, and
(b) detecting the presence or absence of the reaction product as an indication of the presence or absence of isocyanate in the sample. The specific derivatizing agent disclosed is 1-(9-anthracenylmethyl)piperazine (MAP).

PCT WO 99/58517 filed May 13, 1999 by Streicher discloses a compound 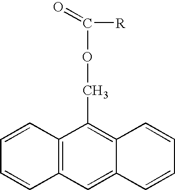
wherein R comprises a radical having a single isocyanate-derivatizing functionality comprising a primary or secondary amine and a process for preparing the compound. The specific compound disclosed is 9-anthracenylmethyl-1-piperazinecarboxylate (PAC). Also disclosed are methods for determining isocyanate in a sample by contacting a sample with the compound of the invention, separating the mixture of ureas from unreacted derivatizing agent, reacting the ureas with sodium thiomethoxide to form 9-anthracenylmethyl methyl sulfide, and quantifying the amount of 9-anthracenylmethyl methyl sulfide produced. A method for measuring isocyanate species is disclosed comprising contacting a sample with the compound of the invention, detecting individual ureas in the sample, and quantifying the amount of urea. The methods of this disclosure are able to determine presence and amount of isocyanate on a surface.

These methods also have drawbacks which will be discussed herein.

It is, therefore, a desire of the present invention to provide a method which is able to derivatize an isocyanate functionality and thereby provide a means by which the presence of isocyanates can be detected both readily and with a high degree of accuracy. Another desire is to provide a means for quantifying the total presence of isocyanate species in a sample, regardless of the particular species present in the sample. A further desire of the present invention is to provide a method wherein the reagents are analytically distinguishable from the reaction product which is analyzed to determine the presence of isocyanates, i.e., the analyte.

These and other desires and advantages of the present invention, as well as additional inventive features, will be apparent from the description of the invention provided herein.

### SUMMARY OF THE INVENTION

In accordance with the purpose(s) of this invention, as embodied and broadly described herein, this invention relates to compounds and methods for the detection and/or measurement of isocyanates.

A method is disclosed for detecting and/or measuring total reactive isocyanate in a sample comprising

a) contacting a bifunctional nucleophilic isocyanate derivatizing agent with a sample, containing or suspected of containing isocyanate, under conditions suitable for the formation of a reaction product capable of detection, and

b) detecting the presence or absence of the reaction product as an indication of the presence or absence of isocyanate in the sample. The bifunctional nucleophilic isocyanate derivatizing agent can have the characteristics of having

1) the two functionalities in the same plane as the molecular backbone, and

2) the two functionalities of the bifunctional nucleophilic isocyanate derivatizing agent being capable of forming a 6-membered ring after binding with an isocyanate group. The bifunctional nucleophilic isocyanate derivatizing agent can be 1,8-diaminonaphthalene (DAN).

Step a) of the method can comprise the steps of
i) derivatizing the isocyanate with the derivatizing agent in the presence of an effective derivatizing catalyst to form an intermediate and
ii) cyclizing the intermediate in the presence of an effective cyclizing catalyst to form the reaction product. A step of eliminating excess derivatizing reagent can be carried out prior to step b).

Also disclosed is a method for detecting and/or measuring total isocyanate in a sample comprising

a) contacting a bifunctional nucleophilic, fused aromatic ring isocyanate derivatizing agent, wherein the two functionalities are amino functionalities in a symmetrical, planar relation to the molecular backbone so as to be capable of forming a cyclic reaction product and capable of reacting with an isocyanate group to form a urea,

with a sample, containing or suspected of containing isocyanate, under conditions suitable for the formation of the cyclic reaction product capable of detection wherein the cyclic reaction product's structure is independent of that of the isocyanate group, and

(b) detecting the presence or absence of the cyclic reaction product as an indication of the presence or absence of isocyanate in the sample.

Further disclosed is a method for detecting and/or measuring total isocyanate in a sample comprising

a) contacting 1,8-diaminonaphthalene with a sample containing isocyanate groups under conditions suitable for the formation of a reaction product capable of detection, and

b) detecting the presence or absence of the reaction product as an indication of the presence or absence of isocyanate in the sample.

Additionally disclosed is a method for determining the species of isocyanate in a sample comprising

a) contacting a bifunctional nucleophilic isocyanate derivatizing agent with a sample, containing or suspected of containing isocyanate, under conditions suitable for the formation of an intermediate capable of detection, and

b) detecting the presence or absence of the intermediate as an indication of the presence or absence of isocyanate species in the sample

A method for determining the total amount of isocyanate on a solid or particle surface is disclosed which comprises

a) contacting a solid or particle surface with a bifunctional isocyanate derivatizing agent under conditions suitable for the formation of a reaction product capable of detection; and

b) quantifying the amount of cyclic reaction product produced. Step a) can further comprise the step of treating the solid or particle surface with a cyclizing catalyst, such as acetic acid, to form a cyclic reaction product.

The compounds of the invention can be used in, for example,
1) a kit for detecting and/or measuring total isocyanate in a sample comprising a bifunctional nucleophilic derivatizing agent;
2) a filter for collecting a sample for detecting and/or measuring total isocyanate in a sample comprising

a) air sample collection filter and

b) bifunctional nucleophilic derivatizing agent; and
3) a kit for solid phase extraction (SPE) for detecting and/or measuring total isocyanate in a sample comprising

a) SPE cartridge and

b) bifunctional nucleophilic derivatizing agent.

The present invention includes a method for detecting the presence of reactive isocyanate groups in a sample by
(a) contacting a bifunctional nucleophilic isocyanate derivatizing agent with a sample under conditions suitable for the formation of a reaction product capable of detection and
(b) detecting the presence or absence of the reaction product as an indication of the presence or absence of isocyanates in the sample.

The present invention also provides for the quantification of the reaction products as an indication of the total quantity of the isocyanate in the sample.

A specific isocyanate derivatizing agent, useful for the determination of isocyanates in a sample, is provided. The agent is 1,8-diaminonaphthalene, referred to herein as “DAN.” DAN has the following chemical structure:

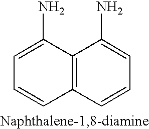

The present inventive methods are useful in the detection and quantification of a variety of isocyanate species in a wide variety of samples and are particularly well-suited to the detection of isocyanates in air or on a surface.

In accordance with the isocyanate detection method, a derivatizing agent, such as DAN or a similar compound, is used in conjunction with any conventional type of environmental sampling device, such as an air sampling device or surface wipe, to contact a sample of isocyanate with the agent. After the derivatizing agent binds with the isocyanate groups forming an intermediate, a cyclization step can be done, and the cyclization reaction product detected.

This method provides a distinct advantage over the isocyanate detection methods currently used by those skilled in the art because all of the isocyanate derivatives yield the same reaction product, and total isocyanate levels are measured by simply measuring the amount of reaction product produced. Alternatively, if measurement of individual isocyanate species is desired, the individual species can be measured by detecting the intermediate formed when the derivatizing agent binds with the isocyanate groups.

A method of the present invention utilizing the DAN reagent circumvents the prior art problems by converting all isocyanate compounds into a single analyte. This analyte can be measured at all levels, well below established isocyanate exposure limits, in the laboratory by a routine, e.g., GC/MS, analysis or in the field, using, e.g., solid-phase extraction followed by fluorescence detection.

A method of the invention also provides an advantage over the prior art by allowing for the detection of isocyanate groups chemically bound to solid or particle surfaces.

The invention provides a rapid, sensitive, inexpensive, and efficient method for the detection and quantification of isocyanates in a sample.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate several aspects of the invention and together with the description, serve to explain the principles of the invention.

FIG. 1 shows the chemical structures of example catalysts for the Step 1 reaction (discussed below) of DAN with isocyanates.

FIG. 2 shows chromatograms of compounds of interest.
A. Chromatogram of a standard mixture used for kinetic studies, each at approximately 1×10⁻⁴ M, showing adequate separation of compounds of interest. This chromatography was used for kinetic studies: 1-perimidone, 2 & 8-impurity, 3-MAP, 4-1,8-DAN, 5-DANBU, 6-DANPU, 7-DANPU, 9-MAPBU, 10-MAPPU.
B. Chromatogram of perimidone from a polymeric MDI sample. Isocratic analysis 23/77 acetonitrile: buffer pH 1.6 at 1.5 mL/min. 2,2-dimethyl-2,3-dihydroperimidine (condensation product of excess DAN with acetone); 1,8-naphthalimide is the internal standard at 1.0×10⁻⁴ M.

FIG. 3 shows spontaneous (non-catalyzed) reaction of butyl isocyanate (1.2×10⁻⁴ M) and phenyl isocyanate (1.1×10⁻⁴ M) with MAP (5×10⁻⁴ M) and DAN (5×10⁻³ M) in acetonitrile and DMSO.
A. Butyl isocyanate reaction with MAP in acetonitrile
B. Butyl isocyanate reaction with DAN in acetonitrile
C. Butyl isocyanate reaction with DAN in DMSO
D. Phenyl isocyanate reaction with MAP in acetonitrile
E. Phenyl isocyanate reaction with DAN in acetonitrile
F. Phenyl isocyanate reaction with DAN in DMSO

FIG. 4 shows reaction half-lives for each combination of eight solvents (Sᵢ, i=1-8) and six catalysts (Cⱼ, j=1-6).

| Sᵢ | Cⱼ |
|---|---|
| S₁ = Acetonitrile | C₁ = N,N,N′,N′-tetramethyl-1,6- |
| S₂ = Dimethyl sulfoxide | hexanediamine |
| (DMSO) | C₂ = Tetrakis(dimethylamino)ethylene |
| S₃ = Dimethyl formamide | C₃ = 1,8-diazabicyclo[5,4,0]undec-7-ene |
| (DMF) | C₄ = 1,3,5-Tris[3-(dimethyl- |
| S₄ = Methylene chloride | amino)propyl]hexahydro-1,3,5-triazine |
| S₅ = Butyl acetate | C₅ = diazabicyclo[2,2,2]octane |
| S₆ = Tributyl phosphate | C₆ = 1-azabicyclo[2,2,2]octane |
| S₇ = Tetramethyl-1,6- | |
| hexanediamine: No reaction | |
| S₈ = 1-methylpyrrolidin- | |
| 2-one | |

Reaction conditions:
DAN=5×10⁻³ M
butyl NCO=1.2×10⁻⁴ M
Solvent Sᵢ (S₁-S₈)
Catalyst Cⱼ=5×10⁻⁴ M
100 μL of the reaction vial was quenched with 900 μL of MAP 5×10⁻³ M

FIG. 5 shows reaction progress of butyl isocyanate with DAN in DMSO at different concentrations of DAN and catalyst C₁, C₅, C₆, and C₇.
A. Catalyst concentration increased 10 times
B. DAN concentration increased 10 times
C. Both DAN and catalyst concentration increased 10 times each
D. C₇at 5×10⁻⁴ M in DMSO
C₁=N,N,N′,N′-tetramethyl-1,6-hexanediamine
C₅=diazabicyclo[2,2,2]octane
C₆=1-azabicyclo[2,2,2]octane
C₇=dibutyltin dilaurate
   Reaction conditions:
DAN=5×10⁻³ M or 5×10⁻² M
butyl NCO=1.2×10⁻⁴ M
Solvent=DMSO
Catalyst Cⱼ=5×10⁻⁴ M or 5×10⁻³ M
100 μL of the reaction vial was quenched with 900 μL of MAP 5×10⁻³ M

FIG. 6 shows spontaneous (non-catalyzed) reaction progress of butyl isocyanate with DAN in five other solvents.
S₉=Dioxane
S₁₀=Di(ethyleneglycol)dibutyl ether
S₁₁=Toluene
S₁₂=Hexamethylphosphoramide: No reaction
S₁₃=Pyridine
   Reaction conditions:
DAN=5×10⁻³ M
butyl NCO=1.2×10⁻⁴ M
Solvent S₉-S₁₃
No catalyst
100 μL of the reaction vial was quenched with 900 μL of MAP 5×10⁻³ M

FIG. 7 shows acid-catalyzed cyclization of DANBU and DANPU (both
1.0×10⁻³ M) in DMSO at 1N (˜10% v/v) and 0.1 N (˜1% v/v) acid.
A. DANBU, 1N acid
B. DANBU, 0.1N acid
C. DANPU, 1N acid
D. DANPU, 0.1N acid

FIG. 8 shows acid-catalyzed cyclization of DANBU and DANPU (both
1.0×10⁻³ M) in DMSO at 10, 20, 30, and 50% AcOH and 100° C.
A. DANPU, 100° C.
B. DANBU, 100° C.

FIG. 9 shows derivatization and cyclization kinetic of 2,4-TDI, 4,4′-MDI, 1,6-HDI, and IPDI monomers with DAN in DMSO. For reaction conditions see the corresponding method section in the Examples.
A. Derivatization
B. Cyclization

FIG. 10 shows the kinetics of the side-reaction perimidone formation from DAN in DMSO and AcOH (1 M perimidone=184 mg/mL solution at room temperature, 5 μl/mL=2.7×10⁻⁵ M).

FIG. 11 shows involvement of DMSO as a catalyst in the derivatization reaction of DAN with isocyanates.
A. Major resonance structures of an isocyanate group
B. Activation of an NCO group by DMSO

a=intermediate RNCO-DMSO complex

b=nucleophilic attack by DAN on the complex

c=urea formation after some steps

FIG. 12 shows
A. Mechanism of acid-catalyzed cyclization of DAN ureas
B. Protonation of the amine group hinders nucleophilic attack on the carbonyl group.

FIG. 13 shows reaction progress of phenyl chloroformate reaction with 1,8-DAN in DMSO.

FIG. 14 shows cyclization kinetic for selected aromatic isocyanates.

FIG. 15 shows DANPU losses in Step 2 (cyclization) of a method of the invention with various ketones.

Reaction conditions:

0.0005M DANPU in 50/50 AcOH/DMSO from 1×10⁻³M DANPU stock in DMSO
Added 3.9 uL K1-Acetone at times tᵢ

4.8 uL MEK

10 uL 2-decanone

10 uL 3-decanone
for an equal concentration of 0.053 M ketone analyzed for perimidone, DANPU-ref used same protocol but 10 uL acetone were added after 1 hr.

FIG. 16 shows DAN consumption with 2-/3-decanone in Step 2 of a method of the invention.
0.005M DAN in 50/50 AcOH/DMSO; +10 uL K7 or K8 and monitored DAN consumption over time.
DAN-V-REF is the reference DAN concentration in the reaction vial @t=0, no ketone.
V=verification of above experiment
0.025M DAN in 50/50 AcOH/DMSO; +5 uL K7 or K8 and monitored DAN consumption over time.
DAN-V-REF is the reference DAN concentration in the reaction vial @t=0, no ketone.
DAN-V/DAN=0.025/0.005=5×
Ki-V/Ki=5/10=0.5×
Overall: 5×0.5=2.5×

### DETAILED DESCRIPTION

Before the present compounds, compositions, articles, devices, and/or methods are disclosed and described, it is to be understood that this invention is not limited to specific synthetic methods, which may vary. It is also to be understood that the terminology used herein is for the purpose of describing particular embodiments only and is not intended to be limiting.

In this specification and in the claims which follow, reference will be made to a number of terms which shall be defined to have the following meanings:

### DEFINITIONS

It must be noted that, as used in the specification and the appended claims, the singular forms “a,” “an,” and “the” include plural referents unless the context clearly dictates otherwise. Thus, for example, reference to “an isocyanate” includes mixtures of isocyanates, reference to “a solvent” includes mixtures of two or more such solvents, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

By the term “effective amount” of a compound or property as provided herein is meant such amount as is capable of performing the function of the compound or property for which an effective amount is expressed. As will be pointed out below, the exact amount required will vary from process to process, depending on recognized variables such as the compounds employed and the processing conditions observed. Thus, it is not possible to specify an exact “effective amount.” However, an appropriate effective amount may be determined by one of ordinary skill in the art using only routine experimentation.

The term “alkyl” as used herein refers to a branched or unbranched saturated hydrocarbon group of 1 or more carbon atoms, such as methyl, ethyl, n-propyl, isopropyl, n-butyl, isobutyl, t-butyl, octyl, decyl, tetradecyl, hexadecyl, eicosyl, tetracosyl and the like.

The term “derivative” is used herein to describe a compound derived from a parent compound. For example, a “derivative of naphthalene” indicates a naphthalene with additional substituents on the naphthalene. As another example, a “derivatized isocyanate” is the resulting compound from a reaction between a derivatizing agent and isocyanate.

The present invention provides a method for detecting the presence of and quantifying reactive isocyanate groups in a sample.

Isocyanates are compounds which contain the functional group —N═C═O and, therefore, have the formula R—N═C═O, wherein R maybe any radical containing at least one carbon atom or H.

A present method comprises
(a) contacting a bifunctional nucleophilic isocyanate derivatizing agent with a sample, containing or suspected of containing isocyanate, under conditions suitable for the formation of a reaction product capable of detection and
(b) detecting the presence or absence of the reaction product as an indication of the presence or absence of isocyanate in the sample.

A present method comprises
(a) contacting a bifunctional nucleophilic, fused aromatic ring isocyanate derivatizing agent,
wherein the two functionalities are amino functionalities in a symmetrical, planar relation to the molecular backbone so as to be capable of forming a cyclic reaction product and capable of reacting with an isocyanate group to form a urea, with a sample, containing or suspected of containing isocyanate, under conditions suitable for the formation of a cyclic reaction product capable of detection wherein the cyclic reaction product's structure is independent of that of the isocyanate group
and (b) detecting the presence or absence of the cyclic reaction product as an indication of the presence or absence of isocyanate in the sample.

While a present inventive method can be used to detect the presence of isocyanate in a sample, the method can also be used to quantify the isocyanate in a sample by quantifying the amount of reaction product.

While other known isocyanate detection methods require separate measurement of each isocyanate species, a method described herein advantageously allows for detection of all isocyanate groups in a single measurement. In accordance with an isocyanate detection method of the present invention, the isocyanate derivatizing agent first binds to the isocyanate species at the isocyanate functional group, −N═C═O. Then, the derivatized isocyanate forms a cyclic reaction product. This cyclic reaction product is the same, regardless of the particular isocyanate species present. Thus, the quantity of cyclic reaction product is equal to the total quantity of isocyanate present in the sample.

Derivatizing Agent

A bifunctional nucleophilic isocyanate derivatizing agent of the present invention can comprise a basic fused aromatic ring structure. The basic fused aromatic ring structure can comprise at least two fused aromatic rings. For example, the at least two fused aromatic rings can be naphthalene. As another example, the at least two aromatic rings can be anthracene. The bifunctional nucleophilic isocyanate derivatizing agent can comprise a compound that is a bi-substituted naphthalene or a derivative thereof Examples of derivatives of a bi-substituted naphthalene include a bi-substituted naphthalene wherein the naphthalene ring(s) have an additional substituent on the ring(s). The derivative of, e.g., naphthalene may be any suitable derivative. It is desirable that any such substituents have relatively small electronic effects in order to minimize any adverse effect they might have on the absorbance or fluorescence properties. Examples of such substituents include alkyls, such as a methyl. Other examples of an additional substituent are ethyl, propyl, butyl, pentyl, hexyl, and the like. Additional substituents can be on the ring(s) as long as they do not change the general reaction(s) desired, as described below, especially if they are located on the opposite side of the agent from the functionalities.

The two functionalities can be isocyanate-derivatizing functionalities wherein the isocyanate-derivatizing functionalities comprise a primary or secondary amine. The two functionalities can each be capable of reacting with an isocyanate group. The two functionalities can each be an amino. The aminos can be in the 1 and 8 positions on the two basic fused aromatic rings (naphthalene). The aminos can be in the equivalent positions on larger rings. An example of equivalents to the 1 and 8 positions of the naphthalene are the 1 and 9 positions of anthracene. The two amino groups can be in the same plane as the molecular backbone, e.g., an aromatic backbone such as naphthalene. The conformation of the bifunctional nucleophilic isocyanate derivatizing agent with the two functionalities can be such that the conformation of the agent forces the, two functionalities (e.g., aminos) to be in the geometric locations (molecular geometry) conducive to cyclization all of the time. The two functionalities are in these locations when they are located to be conducive to Step 2 of the reaction described below (cyclization) once the isocyanate derivatizing agent has reacted with/bound with the isocyanate group(s). The two functionalities (e.g., aminos) can be located in such a way as to be positioned for cyclization of the intermediate from Step 1 derivatization (e.g., urea).

In addition to the characteristic of 1) the two functionalities (e.g., amino groups) being in the same plane as the molecular backbone, it has been found that 2) the ability of the two functionalities bound with an isocyanate group to form a six-membered ring during cyclization is a characteristic of the isocyanate derivatizing agent which can be desirable for the derivatizing agent of the present invention.

The choice of bifunctional nucleophilic derivatizing agent for a particular reaction can be determined by one of ordinary skill in the art, for example, by routine experimentation.

The bifunctional nucleophilic derivatizing agents of the present invention are commercially available or synthesizable by methods known to one of ordinary skill in the art.

The intermediate formed by a derivatization reaction (Step 1 reaction, described below) between a functionality of the derivatizing agent and the C═O of the isocyanate group(s) is a urea when the functionalities are aminos. The urea is capable of cyclization in a Step 2 reaction (described below).

One example of a desirable bifunctional nucleophilic derivatizing agent for the present invention is 1,8-diaminonaphthalene (DAN).

1,8-Diaminonaphthalene (DAN)

DAN, CAS No. 479-27-6, is a bifunctional nucleophilic agent. A bifunctional nucleophile, such as DAN, has two nucleophilic centers capable of reacting with an isocyanate group. The two nucleophilic centers of DAN are aminos.

Further, the functionalities of DAN are located symmetrically in the ring structure(s). This provides for the formation of a single product upon coupling of the amino with the isocyanate group, regardless of which amino (functionality) is bonded to the isocyanate group.

1,8-Diaminonaphthalene (DAN) is commercially available, e.g., Aldrich (Milwaukee, Wis.), or synthesizable by methods known to one of ordinary skill in the art.

Commercial DAN can be purified prior to use. Purification can be done via recrystallization procedures which are known to one of ordinary skill in the art.

DAN can be especially useful for detecting aromatic isocyanates and monomers of aliphatic isocyanates.

The reactivity of DAN was previously always believed to be a high hurdle to overcome in using it as a derivatizing agent. The reaction rate was found to be considerably different in different solvents. For example, the rate of DAN in acetonitrile is much better than in toluene, and the rate in DMSO is much higher than that in acetonitrile (toluene<acetonitrile<DMSO). See the Examples below.

The reactivity of DAN in a DMSO impinger appears to be adequate for sampling aromatic isocyanates. The reactivity of DAN with butyl isocyanate is considerably slower than that for phenyl isocyanate. It is typical that aromatic isocyanates react faster than aliphatic isocyanates. The 3 orders of magnitude in difference with DAN in either solvent (DMSO or acetonitrile) is surprising.

The reaction of solid phase DAN with aliphatic isocyanates is much better than reaction of solution phase DAN, and a DAN-impregnated filter can be used for collection of semi-volatile aliphatic isocyanates.

DAN is well-suited to the use for surface samples, as it is not prone to the limit of detection problems found with other techniques because of the selectivity of detection of the cyclized product.

Derivatization/Reaction Product

The general reaction of a bifunctional nucleophilic derivatizing agent with isocyanate groups proceeds in two steps.

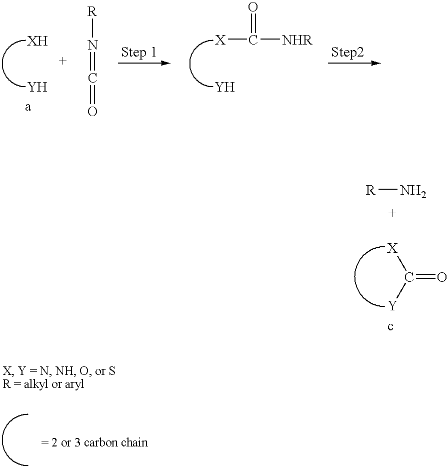

The derivatizing agent, a, can react with all isocyanates in Step 1 to form an intermediate species, b. This reaction can take place upon collection of the isocyanates during environmental sampling. The sample can be analyzed at this stage to enable quantitation of individual isocyanate species of interest (e.g., monomers). The sample containing potentially numerous species b can be treated (chemically, thermally, etc.), resulting in the quantitative transformation of all species, b, to a single reaction product, c. Quantitation of c can serve as a measure of total isocyanate groups.

The intermediate formed by a Step 1 derivatization reaction between a functionality and the —C═O of the isocyanate group(s) is a urea when the functionalities are aminos. The urea is capable of cyclization in a Step 2 reaction. The reactivity of bifunctional nucleophilic derivatizing agents can be a hurdle to overcome in using them as a derivatizing agent with isocyanate. A catalyst can be used to assist the derivatization step. For example, the derivatizing catalyst can be a solvent. One of skill in the art can determine a derivatizing catalyst to use. The derivatizing catalysts of the present invention are commercially available or synthesizable by methods known to one of ordinary skill in the art.

The reaction rate can be considerably different in different solvents. For example, for DAN, the rate in acetonitrile is much better than in toluene, and the rate in DMSO is much higher than that in acetonitrile (toluene<acetonitrile<DMSO). See the Examples below. One of skill in the art can determine a solvent to use for reactions with the chosen bifunctional nucleophilic derivatizing agent. For example, the solvent can be DMSO. For example, the solvent can be a mixture of solvents.

The solvents of the present invention are commercially available or synthesizable by methods known to one of ordinary skill in the art.

The intramolecular cyclization step can also be slow. Catalysis can be used to promote the cyclization step as well. For example, a cyclizing catalyst can be an acid. The acid can be, for example, acetic acid.

One of skill in the art can determine a catalyst to use for cyclization. One of skill in the art can determine an acid to use for cyclization of the intermediate (e.g., urea). See the Examples below. For example, the acid can be a mixture of acids.

The cyclizing catalysts or acids of the present invention are commercially available or synthesizable by methods known to one of ordinary skill in the art.

Reaction of the derivatizing agent, e.g., DAN, with an isocyanate group proceeds in two steps:
(1) derivatization step- derivatizing agent (e.g., DAN) reacts with the NCO group to produce a urea derivative, a reaction analogous to that of all other derivatizing reagents with isocyanates, and
(2) cyclization step-the formed urea undergoes an intramolecular cyclization to produce a cyclic reaction product, e.g., perimidone, whose structure is independent of the parent isocyanate molecule. 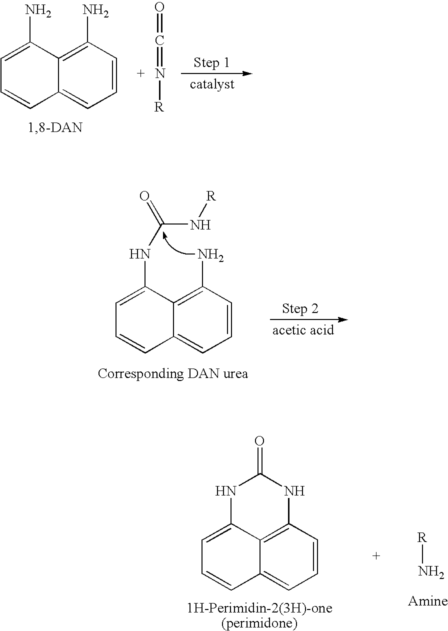
   Molar concentration of the cyclic reaction product, e.g., perimidone, is a direct measure of the total isocyanate group concentration. The second step makes the current scheme unique and fundamentally different from other analytical approaches.

Analogous reactions to the above reactions for DAN would occur for other derivatizing agents of the present invention.

DAN was selected as a very good bifunctional nucleophile candidate based on the cyclization being the limiting step in the formation of the cyclic reaction product.

Acid catalysis can be used in the reaction to promote cyclization. The present invention was able to increase the reaction rate of DAN with isocyanates to make it a viable compound.

Water, that may be encountered in sampling, does not have any effect on the cyclization step, and an appropriate, or effective, amount of DAN on the sampling filter or solution will eliminate isocyanate losses to water or polyols.

As can be seen, the cyclic reaction product is not dependent on the isocyanate species. The cyclic reaction product, e.g., perimidone, is dependent on the derivatizing agent. The amine produced in the second reaction is dependent upon the isocyanate species.

Using any of the derivatizing agents of the present invention, the formation of a derivatized isocyanate (urea) is dependent on the presence of isocyanate in the sample. This, in turn, allows the presence or absence of the derivatized isocyanate or cyclic reaction product to be detected as an indication of the presence or absence of isocyanate in the sample. Moreover, since the quantity of the derivatized isocyanate or cyclic reaction product is directly related to the quantity of isocyanate in the original sample, the quantity of isocyanate can be determined by quantifying the, thus-formed derivatized isocyanate or cyclic reaction product.

An interference is possible between a derivatizing agent and a catalyst which should be addressed in the method. For example, it has been known that a slow reaction between DAN and either acetic acid or some impurity in acetic acid gave rise to the analyte, perimidone. Although perimidone formation from the reaction of DAN with isocyanates is highly selective, this demonstrated that small amounts of perimidone could be generated from other sources. This reaction occurs at a very slow and constant rate. A way found to eliminate the unwanted formation of perimidone was to destroy the excess DAN after the desired Step 2 reaction had taken place. It was found that if a small amount of acetone (or other ketone, see e.g., in Examples) was added to the solution immediately after completion of Step 2, the acetone (or other ketone) rapidly destroys excess DAN and prevents further formation of artifact perimidone.

Alternatively, destruction of excess DAN can be done prior to/during Step 2.

Also, alternatively the excess DAN can be physically removed rather than destroyed.

The DAN-ketone adduct does not interfere with the subsequent analysis of perimidone in the sample either by BPLC or GC. The simple HPLC separation of perimidone and DAN takes advantage of perimidone being a neutral molecule but DAN being a base, the latter being eluted rapidly under acidic mobile phase conditions. Conversion of DAN to its ketone adduct does not compromise this scheme, as the ketone adduct is an even stronger base than DAN itself. It has also been demonstrated that the DAN-ketone adduct is easily separated from perimidone by GC.

Detection of the derivatized isocyanate (urea) or cyclic reaction product can be by any method known in the art.

Method

A present method comprises
(a) contacting a bifunctional nucleophilic isocyanate derivatizing agent with a sample under conditions suitable for the formation of a reaction product capable of detection and
(b) detecting the presence or absence of the reaction product as an indication of the presence or absence of isocyanate in the sample.

A present method comprises
(a) contacting a bifunctional nucleophilic, fused aromatic ring isocyanate derivatizing agent, wherein the two functionalities are amino functionalities in a symmetrical, planar relation to the molecular backbone so as to be capable of forming a cyclic reaction product and capable of reacting with an isocyanate group to form a urea,
with a sample under conditions suitable for the formation of a cyclic reaction product capable of detection
wherein the cyclic reaction product's structure is independent of that of the isocyanate group
and (b) detecting the presence or absence of the cyclic reaction product as an indication of the presence or absence of isocyanate in the sample.

Step (a) of the method can comprise two steps: derivatization and cyclization. These steps are described above.

A method of the invention can further comprise a step of eliminating excess derivatizing agent. The step of eliminating excess derivatizing agent can be prior to step (b) of detecting the presence or absence of the reaction product. This eliminating step can comprise, e.g., adding acetone or other ketone to the reaction mixture after Step 2 cyclization.

The derivatizing agent reacts with the isocyanate functional groups on any isocyanate species present in the sample to form derivatized isocyanates which are a mixture of intermediate ureas.

The mixture of intermediate ureas can then be cyclized and the reaction product treated to eliminate excess derivatizing agent, e.g., DAN, in the reaction mixture. Elimination of excess derivatizing reagent can be achieved by adding ketone, such as 2- or 3-decanone, when the derivatizing agent is DAN. Elimination of excess derivatizing agent can be important when using the present invention to detect the total amount of isocyanates in the sample because the excess derivatizing agent can react with a catalyst, e.g., acetic acid or impurities in acetic acid, to produce excess reaction product.

As indicated above, it has been known that a slow reaction between DAN and either acetic acid or some impurity in acetic acid gave rise to the analyte, perimidone. The best way found to eliminate the unwanted formation of perimidone was to destroy the excess DAN after the desired Step 2 reaction had taken place. If a small amount of ketone is added to the solution immediately after completion of Step 2, the ketone rapidly destroys excess DAN and prevents further formation of artifact perimidone.

Alternatively, rather than destroying excess DAN it can be physically removed. For example, in a field test excess DAN can be removed by SPE to eliminate the potential interference. Solid phase extraction has been investigated to remove excess derivatizing agent. SPE cartridges that have been tried to date include Octadecyl (C-18), phenyl, and mixed mode cation exchange/C-18. The best performance of the 3 tested appeared to be the mixed mode cartridge. If the sampled, which originally contained 50/50 DMSO/acetic acid, was diluted with water, the excess DAN-3-decanone adduct was completely retained while the perimidone was easily washed off the SPE column.

The DAN-ketone adduct does not interfere with the subsequent analysis of perimidone in the sample. HPLC separation of perimidone and DAN takes advantage of perimidone being a neutral molecule but DAN being a base, the latter being eluted rapidly under acidic mobile phase conditions. Conversion of DAN to its ketone adduct does not compromise this scheme, as the ketone adduct is an even stronger base than DAN itself It has also been demonstrated that the DAN-ketone adduct is easily separated from perimidone by GC.

A sample, such as an environmental sample, can be obtained using conventional sampling techniques known to those skilled in the art. The sample can be any environmental sample containing or suspected of containing isocyanates. The isocyanate groups, total NCO concentration, can be detected in various samples, including those of air and surfaces. The method is also able to detect isocyanate present in aerosols. Exemplary environmental samples include, but are not limited to solids, liquids, air, and surface wipe samples. The environmental sample can be an air sample, specifically an air sample from a manufacturing facility that employs isocyanates. The sample can be obtained by any method, for example, by taking discrete samples at periodic intervals.

Sample collection in the present invention can be by any known method. In this respect, a sample, such as air, is contacted with a suitable medium, such as an aprotic organic solvent, which solvent contains the derivatizing agent(s) of the present invention. Each of the isocyanate functionalities present on the isocyanate species react with a single molecule of derivatizing agent under conditions suitable for the formation of derivatized isocyanates, e.g., at ambient temperature and pressure. Typically, impingers or bubblers containing the aforesaid solutions of derivatizing reagents, such as DAN, reagent-coated filters, and reagent-coated sorbents are used as means by which said derivatizing reagents can be exposed to the sample. See, e.g., the Dharmarajan and Purnell articles cited herein, which are hereby incorporated by reference for their teaching on sampling. Filters used in the present invention are generally, for example, 13 mm, 25 mm, or 37 mm in diameter. The filter matrix into which the derivatizing agent can be impregnated is preferably glass fiber or quartz fiber. Air can be generally drawn through the filter with personal sampling pumps, typically at a rate of, for example, about 1 to 2 liters per minute.

For surface sampling, for example, the surface suspected of having isocyanate can be wiped with, e.g., a glass fiber filter moistened with organic solvent. The method of sample collection and the sample will not significantly affect the method. For example, the type of surface wiped can be any surface. Examples are polymeric materials, work space surfaces, and the like. An example of a polymeric material is polyurethane. An example of a work space surface is a wood or wood composite. An example of a particle for solid sampling is wood composite dust created from cutting wood composite made from fiber board and MDI. These particles contaminated with isocyanate can be airborne or on a surface. Other examples of surfaces will be readily apparent to one of skill in the art.

In an embodiment, DAN or a bifunctional nucleophilic derivatizing agent, as provided above, is used to detect isocyanate compounds bound to a solid or particle surface. The solid surface or particles are treated with the derivatizing agent which reacts with the free isocyanate groups on the surface. The solid surface or particles are then treated to generate perimidone from the chemically bound isocyanate groups.

The present method can be used to detect the total amount of isocyanate in a sample or to detect individual isocyanate derivatives. When the present method is used to detect total isocyanate, it is desirable to eliminate excess derivatizing agent because its presence can give an inaccurately high value. Once excess derivatizing agent has been eliminated, this can be followed by detection of the reaction product. When detection of only individual isocyanate derivatives is desired, the entire derivatizing agent-isocyanate derivative may be detected without cyclization and without prior elimination of excess derivatizing agent.

Much like the Streicher WO 99/58517 PAC method, there are 2 chemical reactions involved in the method of the present invention. The first being the derivatization reaction, and the second being a conversion of the isocyanate derivative(s) to a single product analyte. The 2 chemical reactions in the PAC method are derivatization of the isocyanate and cleavage of the analyte from the derivatized isocyanate.

Even though DAN's reaction with isocyanates as measured in acetonitrile solution is orders of magnitude slower than established reagents, the potential for interferences in the method are lower, as the analyte actually contains a part of the isocyanate group, i.e., the selectivity is better.

The detection and quantification of the reaction product, i.e., derivatized isocyanates or cyclic reaction product, may be performed in any suitable manner. The present invention allows for the very reliable detection and quantification of the isocyanate reaction products resulting from the use of an isocyanate derivatizing agent in accordance with the present invention.

Analysis of the reaction product, e.g., perimidone, can be performed with standard instrumentation, for example, HPLC/fluorescence, HPLC/UV, or GC/MS. This analysis is very simple. Selection of a method of analysis of the reaction product can be readily determined by one of ordinary skill in the art. The instrumentation is commercially available.

The mass spectrum of perimidone is well suited to routine GC/MS monitoring. The mass spectrum consists primarily of the molecular ion (184) and a rather unique fragment ion (166). Quantification can be based on the response of the 184 ion, and the abundance of the ratio of 184/166 can serve as a quality control check that the measured signal is coming completely from perimidone. One of the advantages of GC/MS is that any response variability can be largely corrected for by using a deuterated internal standard. In theory, the deuterated internal standard (which is perimidone with some hydrogens replaced with deuterium atoms) will undergo the same response changes as the analyte perimidone, so that quantification is accurately accomplished using the ratio of response of analyte to internal standard.

The internal standard can be added to the sample at the same time acetone is added to destroy the excess DAN or any time prior to GC/MS analysis.

The specificity of GC/MS enables simple and accurate measurements with the difficult surface sample matrices.

Another possibility is supercritical fluid chromatography (SFC)/MS. This method could handle analytes that were too non-volatile or too thermally fragile for GC, but provided better resolution and better compatibility with MS than HPLC.

The method(s) of the present invention can be used in a laboratory setting or in a field setting.

Laboratory analysis of perimidone can be done, e.g., by BPLC with fluorescence detection. The method is quite sensitive and fairly selective. A lab test using a bifunctional nucleophilic derivatizing agent, e.g., DAN, has an advantage of high sample throughput and almost no interpretation required by the analyst.

A field kit containing bifunctional nucleophilic derivatizing agent, e.g., DAN, can provide a quantitative measure of total isocyanate group. For field use, the compatibility between Steps 1 (derivatization) and 2 (conversion to single analyte) is important. If the solvent used in Step 1 (either as impinger solvent or solvent used to extract the filter after sampling) is incompatible with Step 2, it has to be removed prior to carrying out Step 2. This can be incredibly difficult in the field, and is at least very undesirable as an extra step in the lab or the field. However, in the current method, the specific solvent that enhances the activity in Step 1 (DMSO) is very compatible with Step 2. Simply adding an equal volume of acetic acid to the DMSO solution from Step 1 enables Step 2 to take place rapidly for aromatic isocyanates.

In-field analysis has the advantage of giving results shortly after sampling. The connection can be made more easily between a specific work task and the resulting exposure. The major analytical compromises that must be made to implement in field methodology are the loss of high resolution chromatography and the possible use of MS detection. The separation technique can be solid phase extraction which is a low resolution chromatography technique. An advantage that the DAN/perimidone system has with SPE is that potentially all interfering compounds in a sample will be basic in nature. By using cation exchange SPE or possibly even reversed phase SPE with a low pH mobile phase, all of these potential interferents can be successfully separated from perimidone. Selective elution of the perimidone can be followed by detection with a portable fluorescence spectrophotometer.

The current method has several advantages over other methods that seek to measure total isocyanate exposure. Its primary advantages are the simplicity of analysis and versatility of its use. The method is complementary to established HPLC methods for total isocyanate, providing a simple measure of total isocyanate. A speciation of that isocyanate is not as simple as in, for example, NIOSH Method 5525 or MDHS 25/3. However, in many environments, it would be a desirable alternative to these HPLC methods. DAN-isocyanate samples sent to the laboratory can be analyzed routinely with very short analysis times (estimate 10 minutes per analysis). The results require very little interpretation. By contrast, current HPLC methods typically require analyses lasting 30 minutes, and the results require interpretation by an experienced analyst. Another option for use would be as a field screening technique where the results are obtained on site within minutes of sample collection. A field screening technique could not rely on sophisticated instrumentation such as GC/MS. However, solid-phase extraction (SPE) of the sample followed by fluorescence spectrophotometry can be accomplished in the field. The DAN method can be amenable to both air and surface samples. It can be applied to measuring free isocyanate group bound to particles or residual isocyanate groups present on bulk polyurethane foam, applications where conventional HPLC methods would not work. The Streicher PAC method can also be used for this type of sample, but the greater selectivity of the DAN method makes it a better choice. Potential commercial products associated with the use of this method include DAN-impregnated filters for air sampling, kits containing solutions for the two steps of the derivatization reaction, or cartridges for removal of excess reagent in the field before analysis.

The current method is similar to the Streicher PCT WO 99/58517 PAC method in that each method seeks to accomplish measurement of the isocyanate(s) by converting the complex isocyanate mixture into a single analyte species for easy measurement. Both methods are capable of measuring individual monomer species, particularly diisocyanate monomers.

The previous Streicher U.S. Pat. No. 5,354,689 MAP method seeks to measure the mixture by enabling correct identification and accurate quantification of the individual isocyanate species. This method, however, was not capable of measuring certain species, such as those isocyanate groups chemically bound to particles.

A bifunctional nucleophilic method has advantages, to varying degrees, over previous methods, such as tryptamine, MAP, MDHS 25, and MAMA methods: 1) the previous methods must assume that the reagent reacts only with isocyanates, as all reagent-derived chromatographic peaks are quantified as isocyanate. In contrast, very few compounds other than isocyanates could react with the bifunctional nucleophile to produce the derivative that is quantified in the current method. This makes false positives much less likely. 2) The previous methods require that all compounds elute as observable BPLC peaks. In the bifunctional nucleophilic method, quantitation involves integration of a single chromatographic peak. 3) In the previous methods, it must be assumed that all isocyanates derived from a particular monomer have the same detector response factor. The detector response factors of several tryptamine-derivatized isocyanates have been found to vary with a standard deviation of 16%. Because there is only a single derivative in the bifunctional nucleophilic method, there is no possibility of structure-related differences in response factor. 4) The previous methods require two detectors for the confirmation of the identity of peaks as derivatized isocyanates. Because chromatographic retention time can be used for identification in the bifunctional nucleophilic method, use of a single detector is sufficient.

As stated previously, the reaction product/derivatized isocyanate possesses the ability to be detected at low levels by conventional apparatuses, e.g., using HPLC with UV or fluorescence.

The compounds of the invention can be readily synthesized using techniques generally known to synthetic organic chemists. Suitable experimental methods for making and derivatizing aromatic compounds are described, for example, in the references cited in the Background section herein above, the disclosures of which are hereby incorporated by reference for their general teachings and for their synthesis teachings. Methods for making specific and preferred compounds of the present invention are described in detail in the Examples below.

### EXAMPLES

The following examples are put forth so as to provide those of ordinary skill in the art with a complete disclosure and description of how the compounds, compositions, articles, devices, and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric.

### Example 1

### Catalysis of DAN-Isocyanate Derivatization Reaction

The purpose of this example was to identify successful combinations of solvent and catalyst for the reaction of 1,8-diaminonaphthalene (DAN) with aliphatic isocyanates at ambient temperature in order to achieve reaction rates comparable to those of commercial reagents.

The 2-step reaction scheme between DAN and isocyanate is shown above.

Isocyanates

Two monoisocyanates, butyl and phenyl isocyanate, were used as the model isocyanates to study derivatization rate with DAN. The main advantage of using a monoisocyanate was simplicity in monitoring reaction products and interpreting kinetic results with regard to the behavior of solvent, catalyst, and the isocyanate structure-NCO reactivity interplay.

Concentrations

From preliminary tests in acetonitrile, concentrations of 5×10⁻³M and ˜1×10⁻4M for DAN and monoisocyanate were selected, respectively. For comparison purposes, reaction kinetics of MAP (5×10⁻⁴M) with butyl and phenyl isocyanate (˜1×10 ⁻⁴M) in acetonitrile was also studied.

Three factors were selected for studying the derivatization reaction of DAN and isocyanates: the isocyanate structure, the solvent, and the catalyst.

Reagents

All reagents, of the highest purity available, were purchased from Aldrich (Milwaukee, Wis., unless otherwise specified.

Originally, 8 polar aprotic solvents and 6 tertiary amine catalysts were chosen.

Solvents were selected on previously defined criteria, such as chemical inertness to isocyanates or DAN, good solubility of reactants and reaction products.

These solvents were acetonitrile (99.5+%), dimethyl sulfoxide (DMSO)(99.9%), N,N-dimethylformamide (DMF) (99.8%), dichloromethane (99.9%), butyl acetate (99.5+%), tri-n-butyl phosphate (TBP)(99+%), tetramethyl-1,6-hexanediamine (THDA)(99%), and 1-methylpyrrolidin-2-one (MP)(99+%).

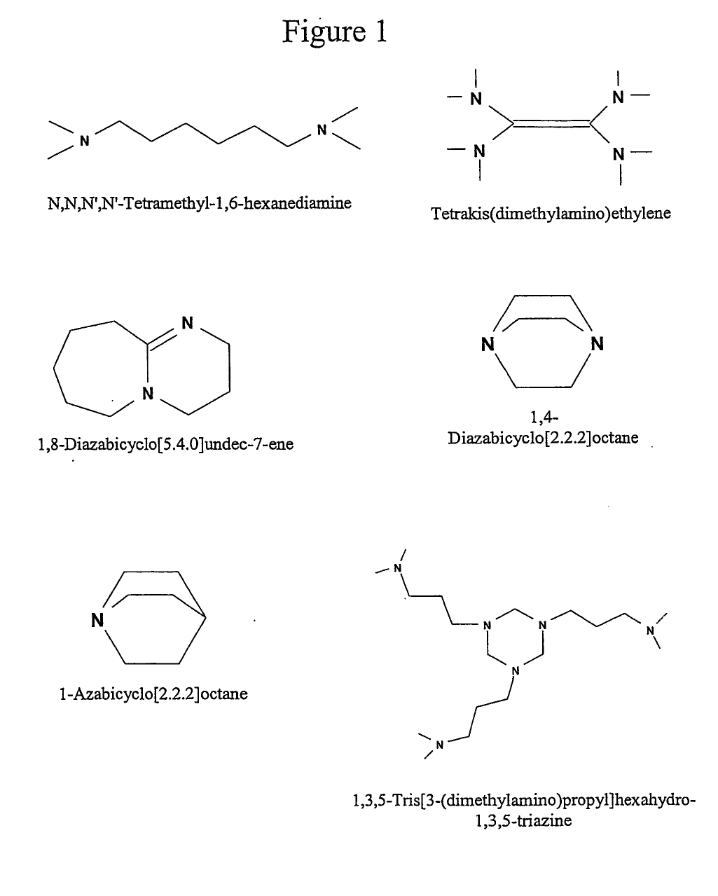

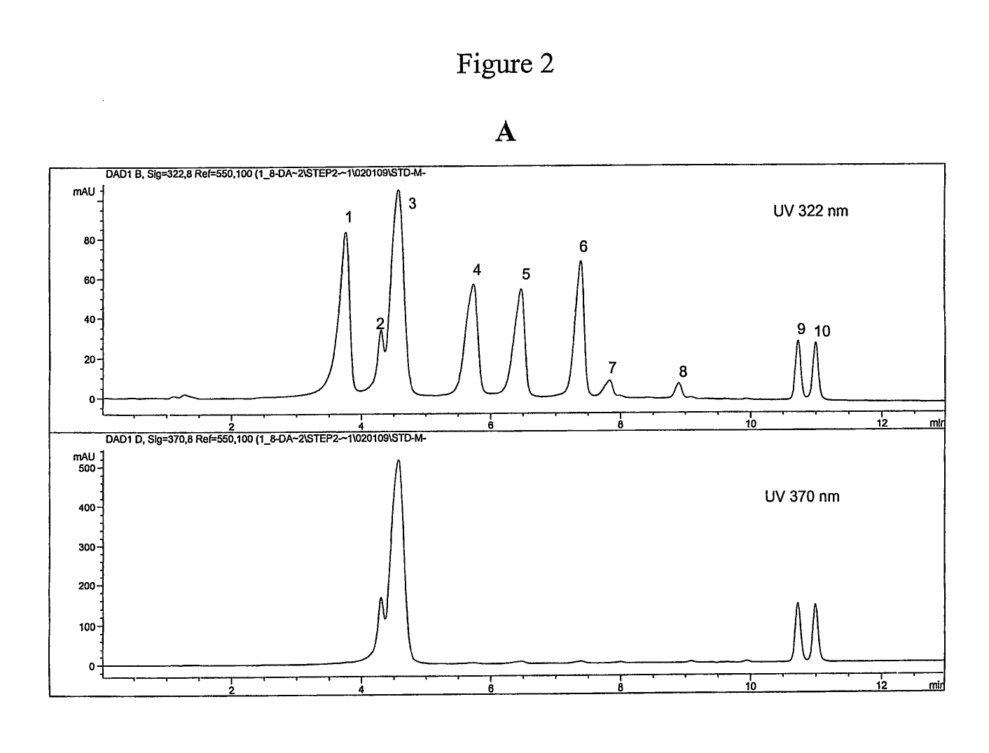

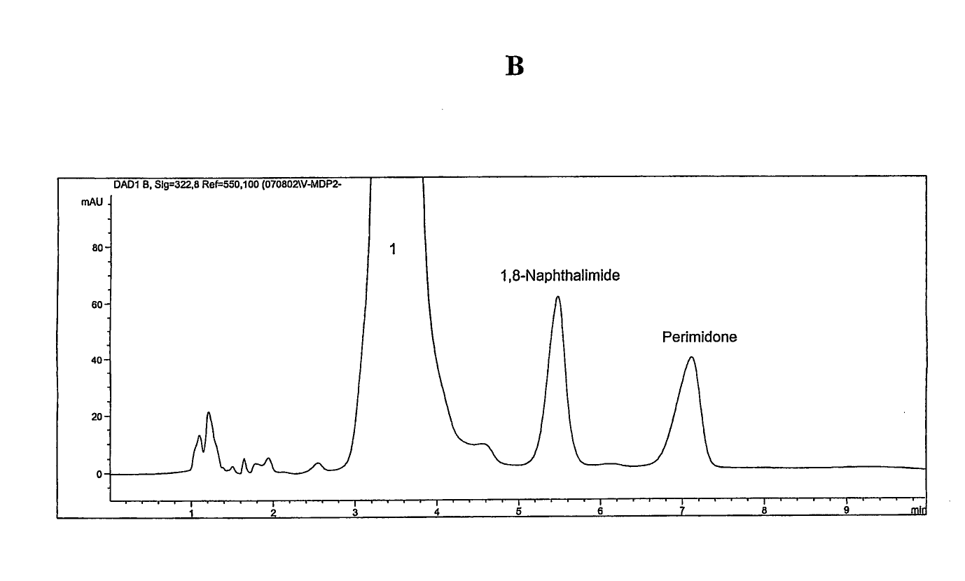

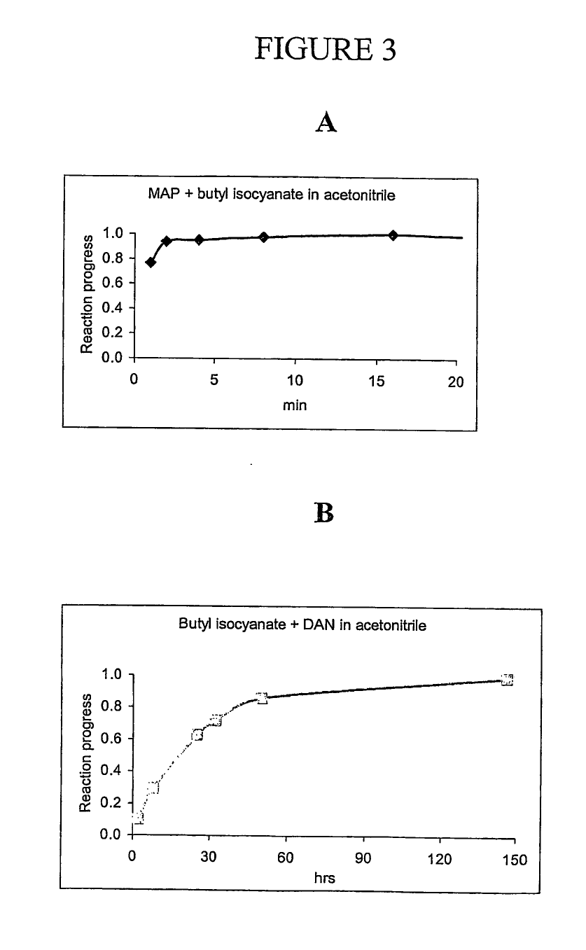

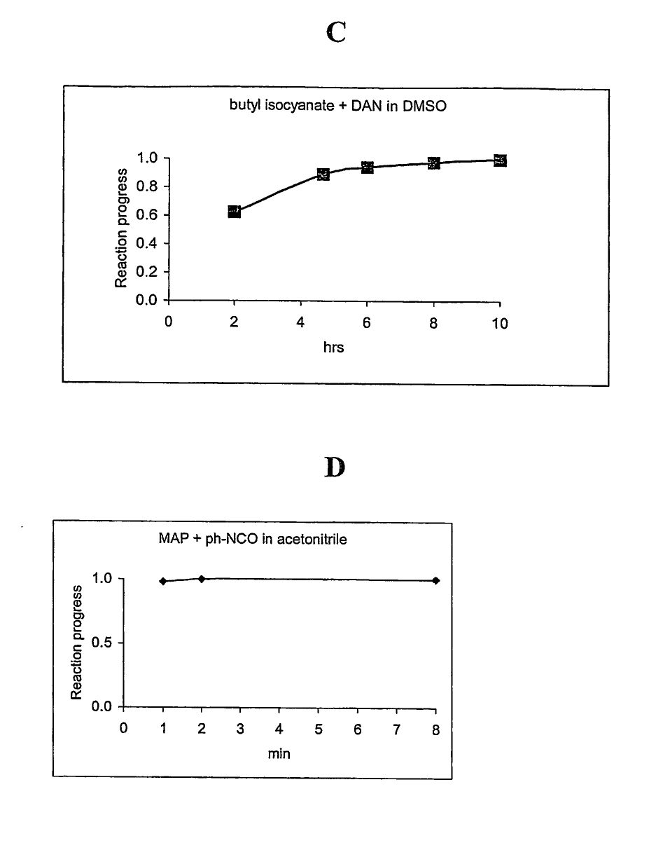

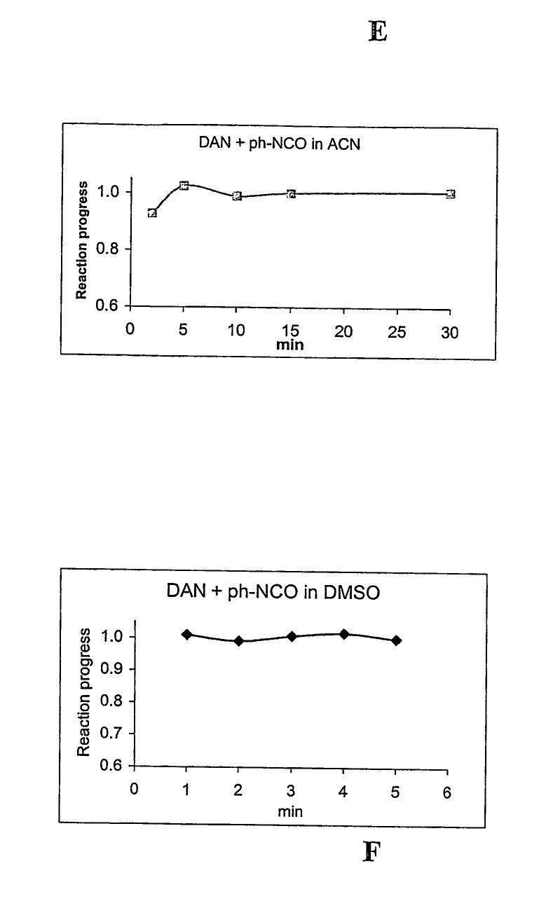

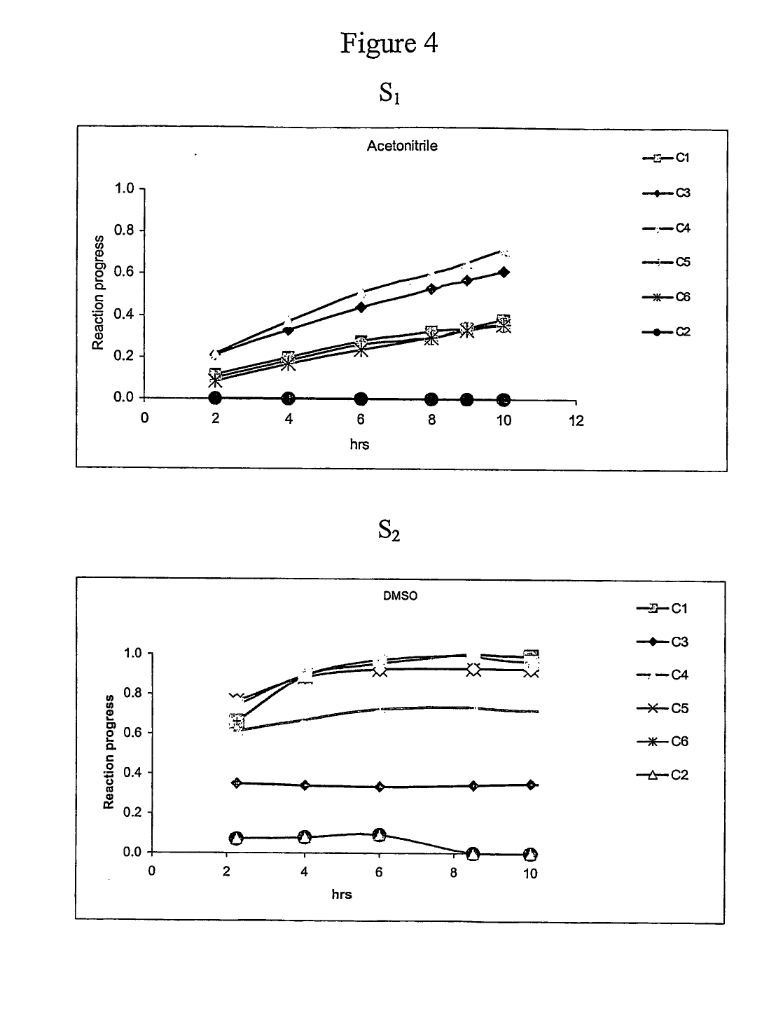

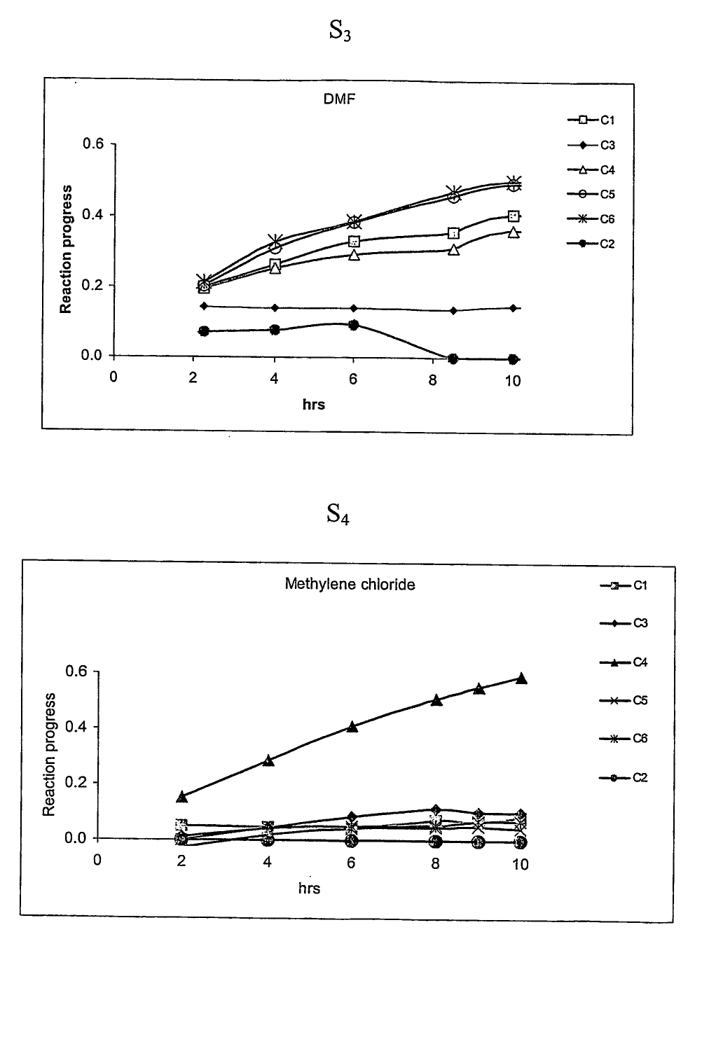

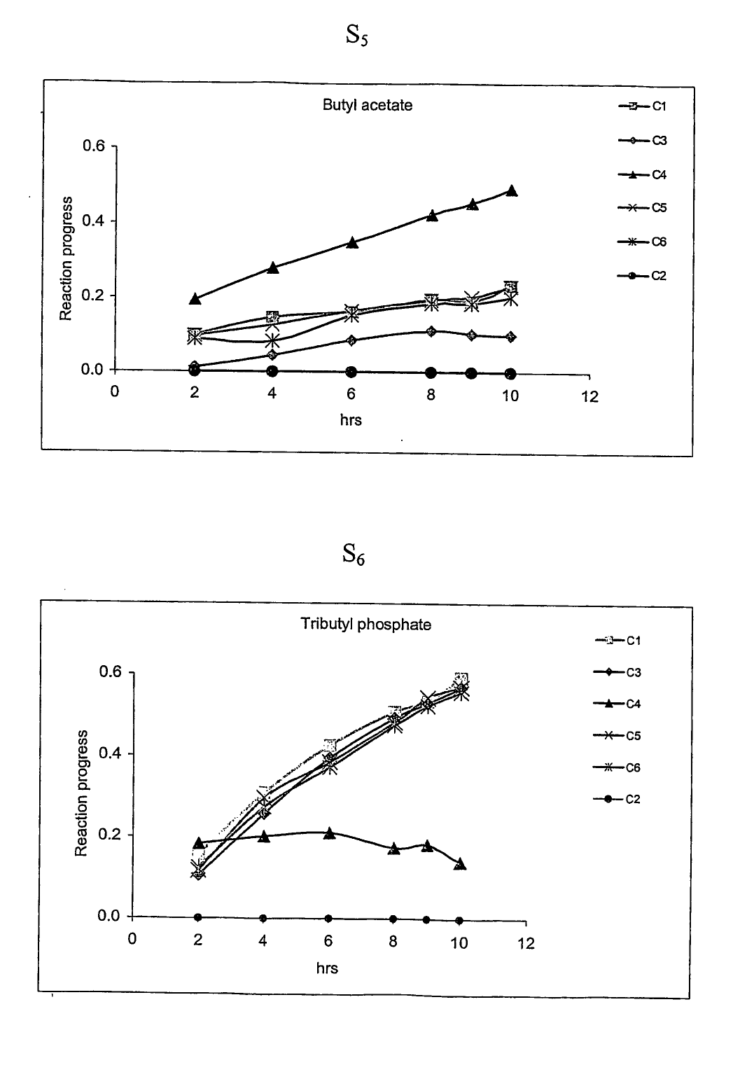

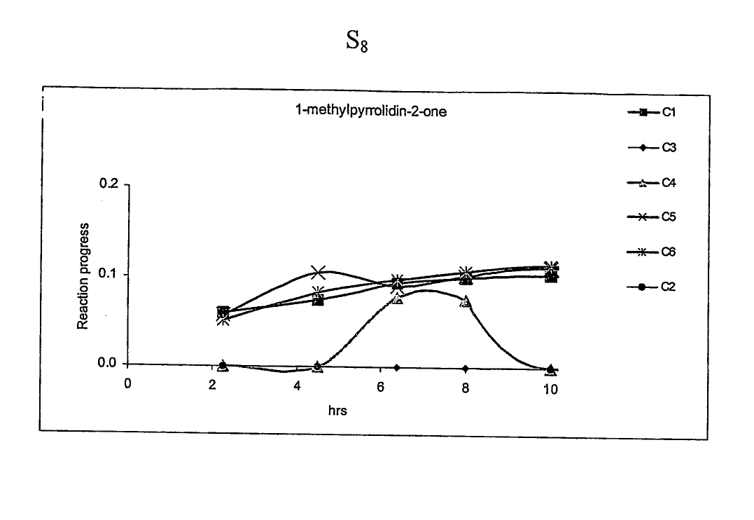

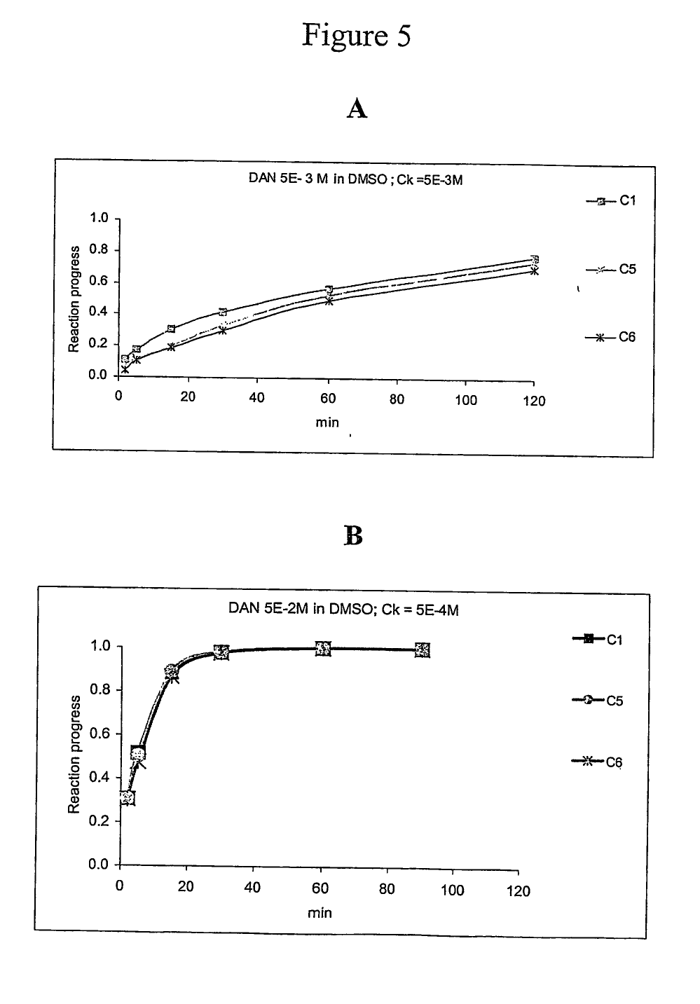

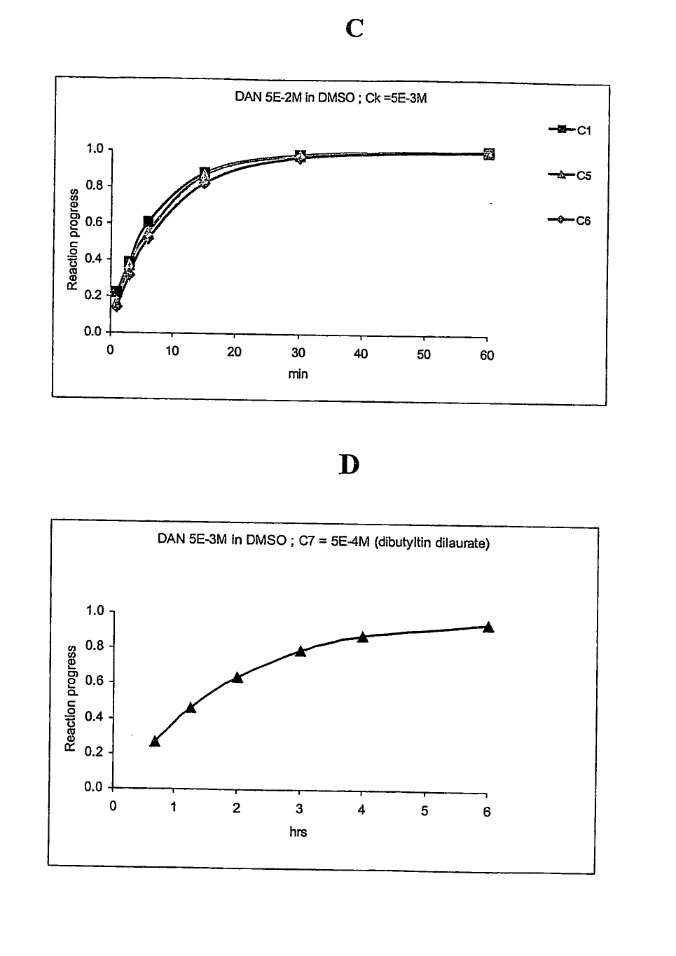

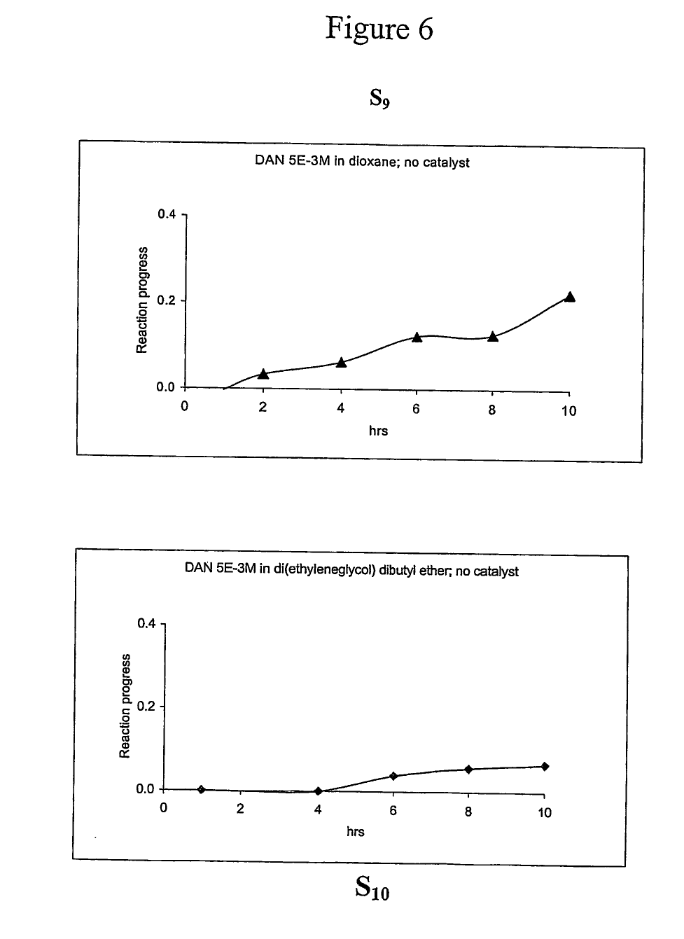

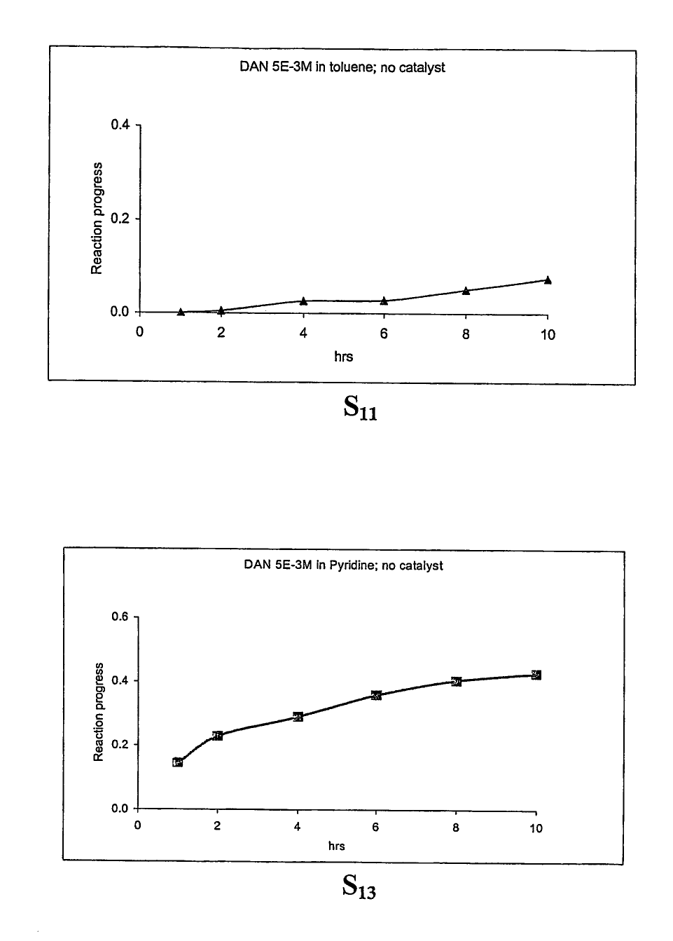

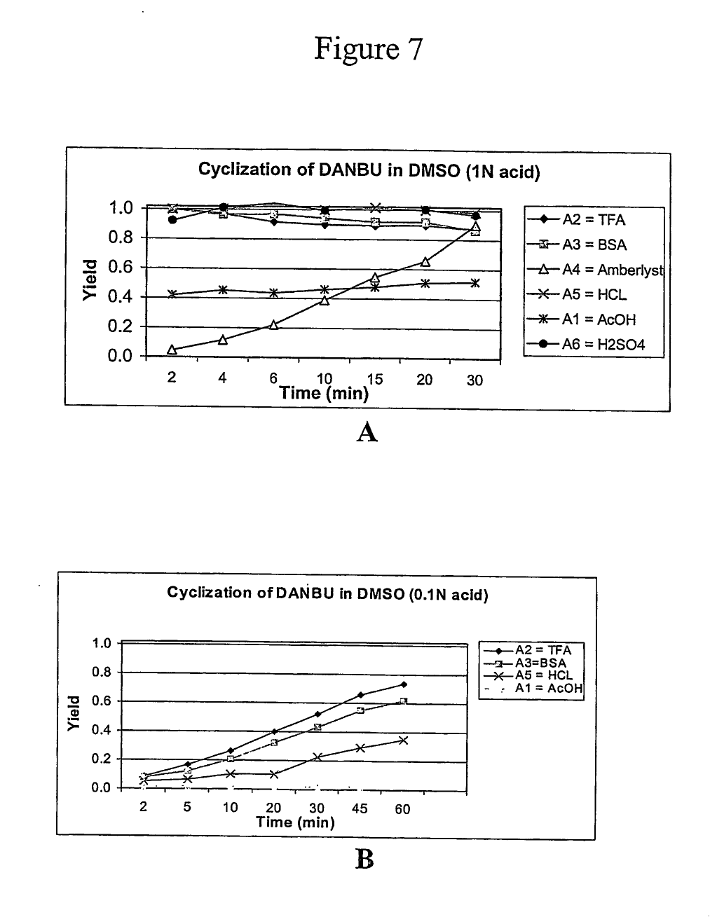

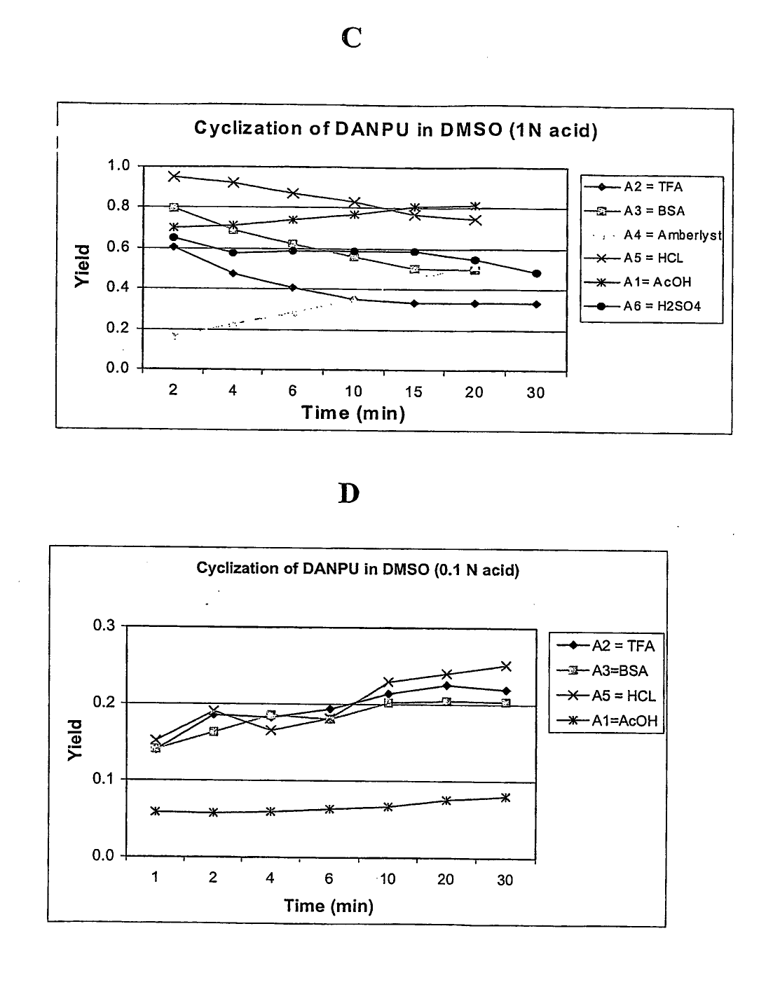

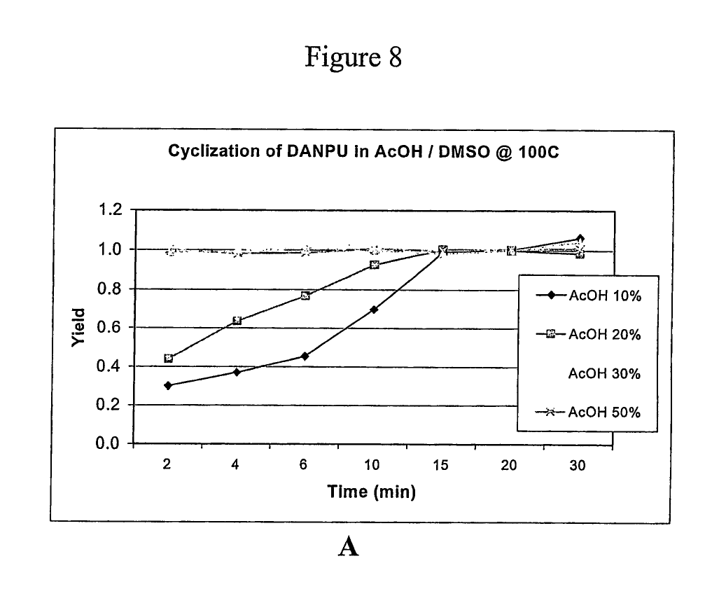

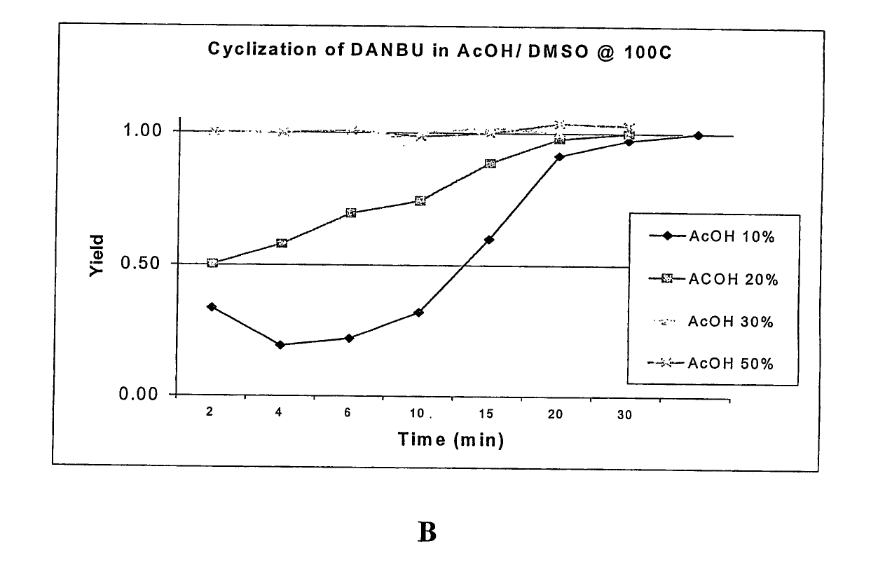

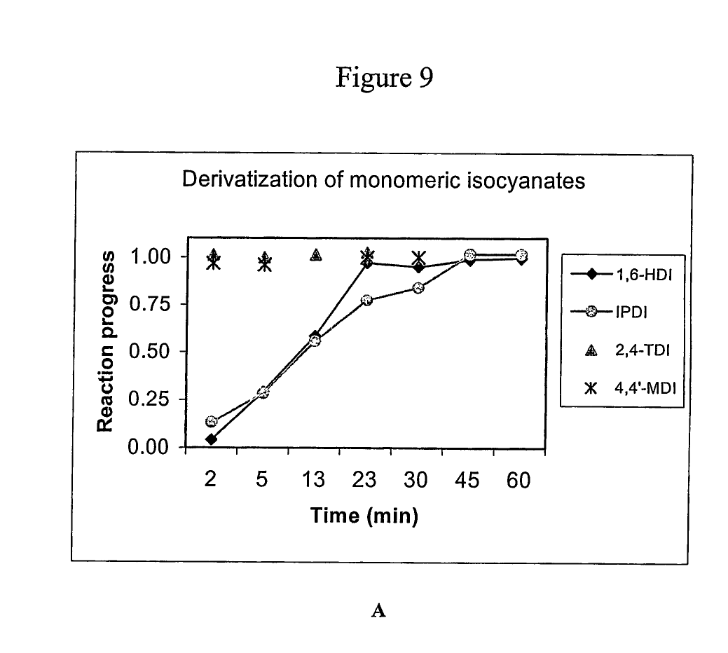

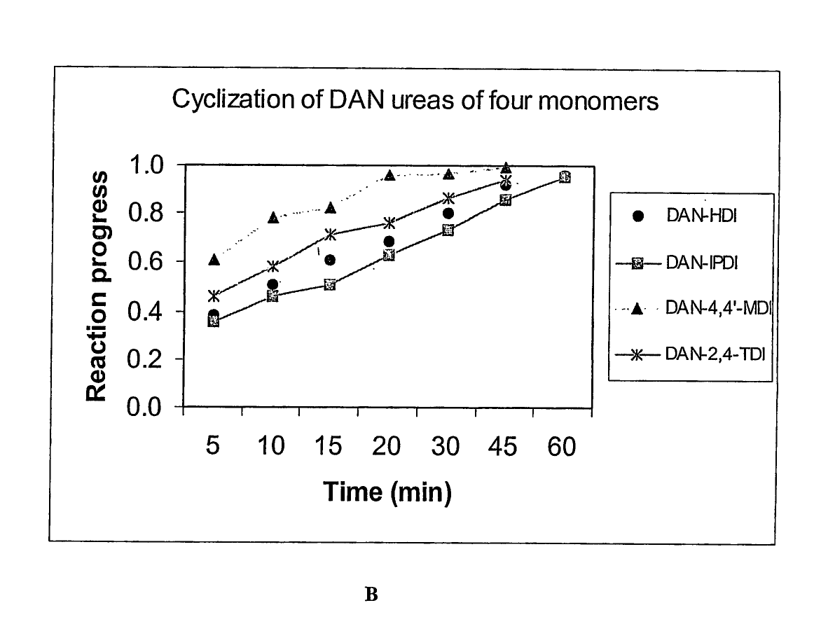

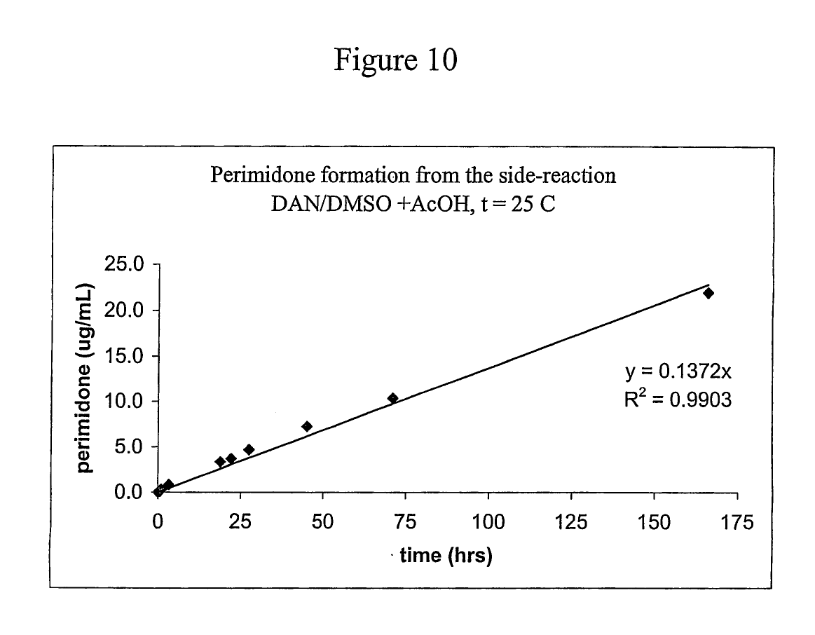

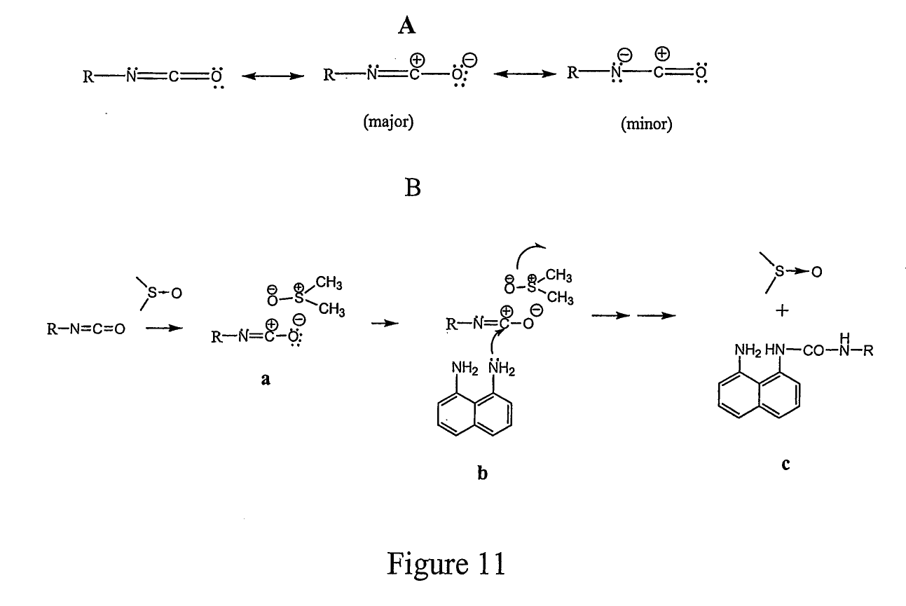

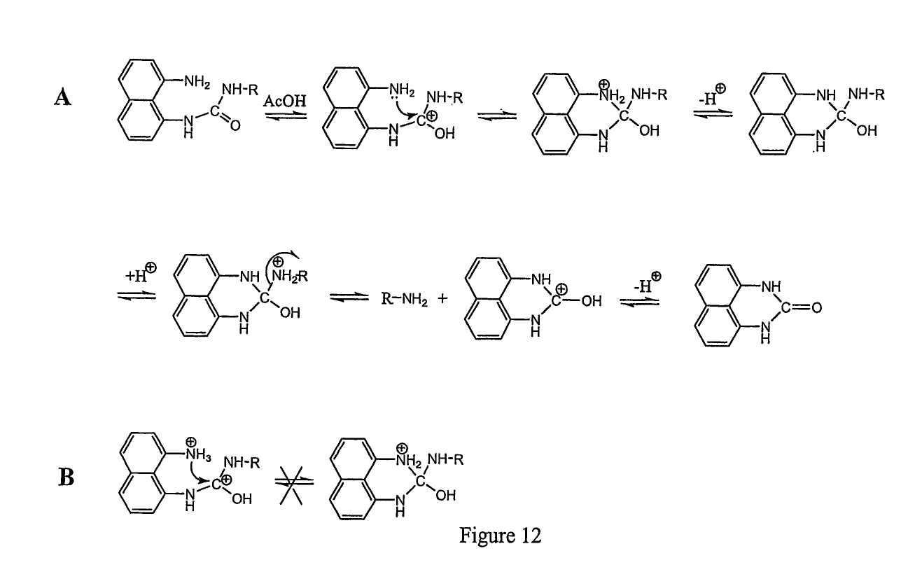

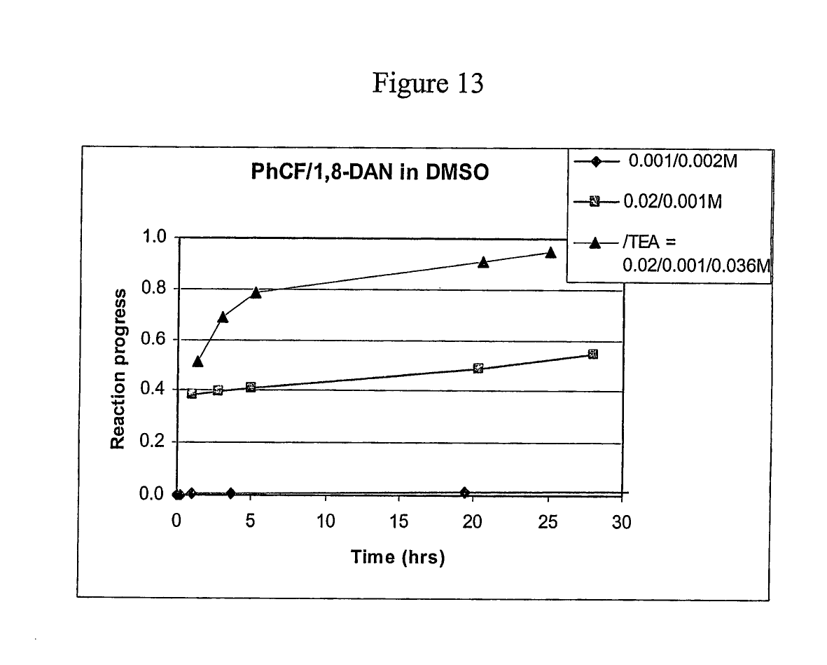

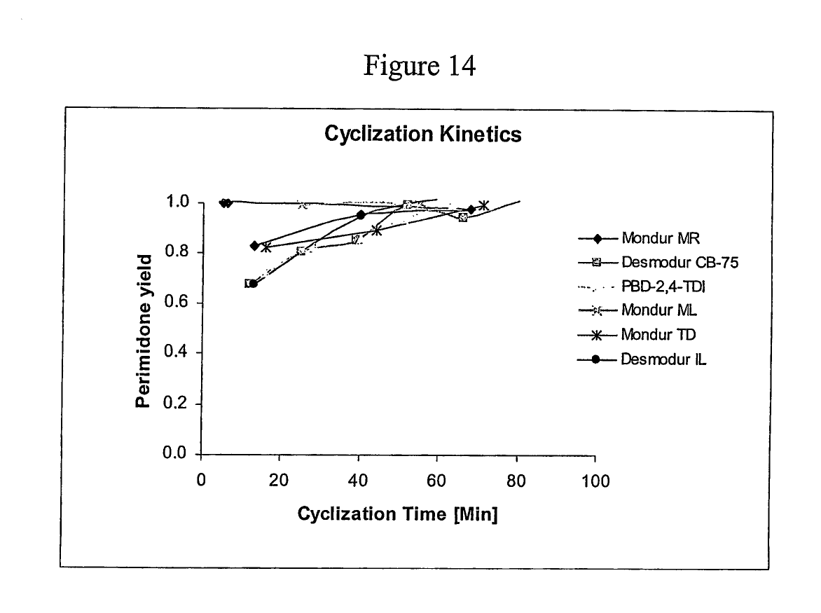

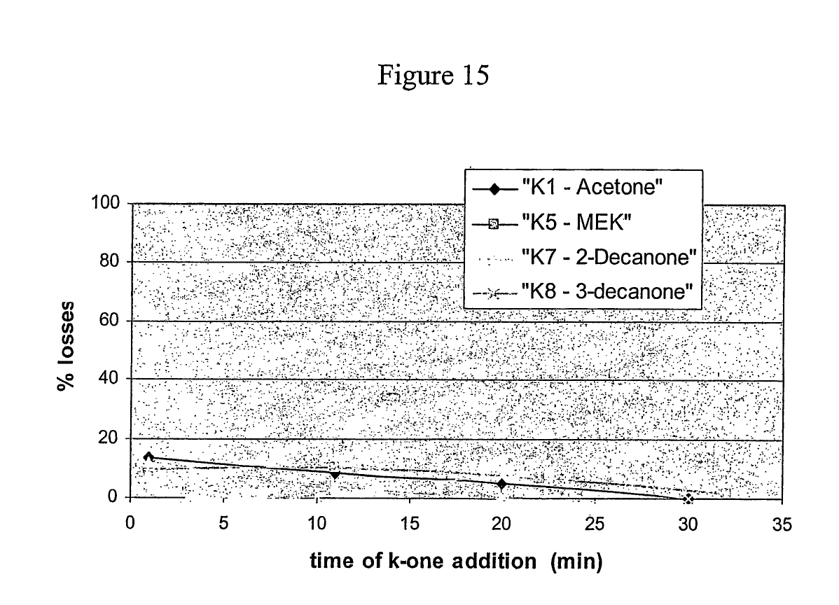

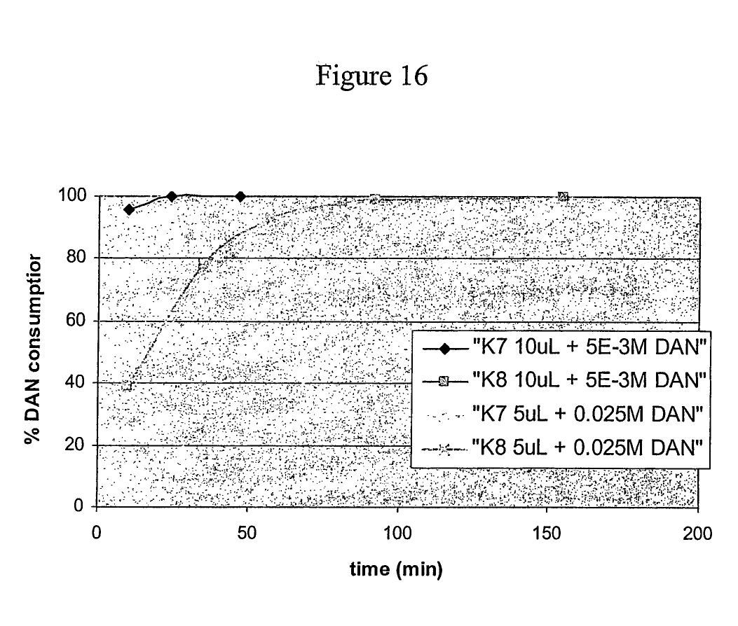

Six catalysts (FIG. 1), chosen from the published literature as promising basic tertiary amine catalysts, were tetramethyl-1,6-hexanediamine (C₁)(99%), tetrakis(dimethyl amino)ethylene (C₂)(95%), 1,8-diazabicyclo[5,4,0]undecene-7 as its 1:1 salt with 2-ethylhexanoic acid (C₃)(98%), 1,3,5-tris[3-(dimethylamino)propyl]-hexahydro-1,3,5-triazine (C₄)(98%), 1,4-diazabicyclo[2.2.2]octane (DABCO) (C₅)(98%), and 1-azabicyclo[2.2.2]octane(quinuclidine) (C₆)(97%).

Later, 5 more solvents (toluene 99.5+%, hexamethylphosphoramide 99%, 1,4-dioxane 99+%, di(ethylene glycol) dibutyl ether 99+%, and pyridine 99.9+%) and one catalyst (dibutyltin dilaurate 95%) were individually tested.

Commercial grade DAN (specified purity 99%), known to be unstable and light sensitive, was found by HPLC to be only 93% pure. After recrystallization from boiling hexane, DAN purity increased to 99.8+%.

1-(8-aminonaphthyl)-3-butylurea (DANBU), 1-(8-aminonaphthyl)-3-phenylurea (DANPU), and 1H-perimidin-2(3H)-one (perimidone) were previously synthesized and characterized. DAN was reacted with butyl or phenyl isocyanate, respectively, at room temperature and pressure in a solvent having good solubility and reactivity, diethyl ether. A volume reduction was performed and the ureas precipitated. The phenylurea was dissolved in acetic acid to form the perimidone. Crystals of the perimidone were recovered during a volume reduction.

The DANBU and DANPU purity prior to use was found by HPLC to be 99.7+ and 90%, respectively. DANBU and DANPU appeared to be very stable. Perimidone was further characterized prior to step 2 cyclization experiments. Perimidone, which was obtained as crystals after precipitating from a solution of DANPU in glacial acetic acid, was found to be a complex perimidone.AcOH, with one AcOH molecule in the structure. Perimidone of very high purity was obtained by sublimation of the perimidone.AcOH product at 190° C. and 30 mtorr. The identity and purity of perimidone.AcOH and perimidone were confirmed by elemental analysis and NMR. Elemental analysis of purified perimidone gave C 71.75%, H 4.35%, and N 15.14%, which are in excellent agreement with the theoretical values. Pure perimidone is a white, odorless powder, whereas periinidone.AcOH is a grayish needle-shaped solid, with a strong acetic acid smell.

Perimidone is very stable under the various conditions tested.

Absorbance/Fluorescence

The UV absorbance properties of DAN, DANBU, DANPU, and perimidone were previously studied. All four compounds exhibit similar UV characteristics. They have a maximum absorbance at around 200 nm, and at least two weaker bands at around 230 and 322 nm. The 230 and 322 nm bands were found to be about 1.4 and 4.7 times weaker than the 200 nm band, respectively.

Fluorescence properties of perimidone were very similar to that of DAN. The fluorescence did not appear to offer any significant sensitivity advantage over UV for monitoring DANBU. Both DAN ureas and MAP ureas were monitored at their more specific wavelengths of 322 nm and 370 nm, respectively.

Instrumentation

An Agilent HP1100 HPLC system consisting of a quaternary pump, vacuum degasser, autosampler, a diode array detector (DAD) and a fluorescence detector (FLD) were used for analysis. The whole system was controlled through the Agilent Chemstation software. The chromatographic column was an Inertsil C8 MOS (monooctyl silane) PEEK 150×4.6 mm, 5 μm particle size from Phenomenex (Torrance, Calif.). The guard column was a 4×3 mm SecurityGuard® (Phenomenex, Torrance, Calif.). Column temperature was maintained constant at 30° C. Separation of DAN, DAN ureas (DANBU, DANPU), and MAP butyl isocyanate urea (MAPBU) was achieved with the gradient at a flow rate of 1.5 mL/min—from 30% acetonitrile/70% triethyl ammonium phosphate/formate buffer at pH 6.0 to 40/60 in 4 min., to 95/5 from 4 to 13 min., followed by 5 min. column re-equilibration at 30/70. FIG. 2A is a chromatogram of a standard mixture illustrating adequate resolution of compounds of interest during the derivatization kinetic studies.

Experimental Design for Evaluation of the Derivatization Kinetics

Reaction kinetics were originally monitored for all combinations of the first 8 solvents and 6 catalysts. One mL of equimolar concentrations of 5×10⁻⁴M each DAN and MAP were allowed to react competitively for a limited amount of 1.2×10⁻⁴M butyl isocyanate for over 24 hrs.

Quenching of the MAP and butyl isocyanate reaction was achieved by transferring 100 μL of the solution into a 2 mL vial containing 900 μL of 0.01M dibutylamine (DBA) in acetonitrile.

Reactions of DAN and butyl isocyanate for each solvent-catalyst combination were carried out in a 2 mL vial by transferring 1.6 mL of 5×10⁻³M stock DAN solution in each solvent, 8 μL of 0.024 M butyl isocyanate stock solution in methylene chloride, and 2 μL of stock catalyst solution in methylene chloride. Concentration of reactants in the reaction vial was 5×10⁻³ M DAN, 1.2×10⁻⁴ M butyl isocyanate, and 5×10⁻⁴ M catalyst. From each reaction vial an aliquot of 100 μL was transferred at appropriate time intervals to a quenching vial containing 900 μL of 5×10⁻³M 1-(9-anthracenylmethyl)piperazine (MAP) in acetonitrile. Typical sampling times were 2, 4, 6, 8, 10, and 24 hrs.

Similarly, the non-catalyzed (or spontaneous) reaction of phenyl isocyanate (1.1×10−4 M) with MAP (5×10−4 M).in acetonitrile, and with DAN (5×10−3 M) in acetonitrile and DMSO was investigated following identical procedures as described for butyl isocyanate. Aliquots were taken from the reaction vials at appropriate time intervals during 8 min. for MAP, 120 min. for DAN in DMSO, and 180 min. for DAN in acetonitrile.

Concentration of butyl and phenyl isocyanate in the stock solutions was independently determined from its direct derivatization with MAP using the calibration curve of MAP hexamethylene diisocyanate (HDI) urea. All reactions were carried out at the typical laboratory temperature of 22 to 25° C.

Experimental Results for the Derivatization Step

The reaction of 5×10⁻⁴ M MAP with 1.2×10⁻⁴ M butyl isocyanate in acetonitrile proceeded very rapidly (FIG. 3). The reaction was completed in about 10 minutes with a half life (t_{1/2}) of <1 min. (77% complete in 1 minute). In contrast, the similar reaction of DAN with butyl isocyanate was extremely slow, with an estimated t_{1/2} on the order of one week. At 5×10^{31 3} M DAN reaction was completed in about 150 hrs (t_{1/2}˜14 hrs) in acetonitrile and 10 hrs (t_{1/2}˜1 hr) in DMSO. DAN reactivity is almost ten thousand times slower than MAP. This explains the fact that in the competitive reaction of equimolar concentrations of MAP and DAN for a limited amount of butyl isocyanate only MAPBU was found.

The spontaneous reaction of 1.1×10⁻⁴ M phenyl isocyanate with 5×10⁻⁴ M MAP in acetonitrile and 5−10⁻³ M DAN in DMSO was completed in one minute or less and could not be measured (FIG. 3).

Similarly, the phenyl isocyanate reaction with DAN in acetonitrile was completed in about 5 min. The same reaction of phenyl isocyanate with 5×10⁻⁴ M DAN was completed in 1-2 min in DMSO and 3540 min in acetonitrile. Phenyl isocyanate losses of 50% and 5% were observed at this lower DAN concentration for the reaction in DMSO and acetonitrile, respectively. The data suggest that the reaction of phenyl isocyanate with 5×10⁻³ M DAN in DMSO is completed in <20 sec.

FIG. 4 provides kinetic data on the catalyzed reaction of DAN and butyl isocyanate. The graphs show a strong solvent effect in the reaction kinetics. Only DMSO performed satisfactorily and noticeably better than all other solvents. The best t_{1/2} l in DMSO for 3 catalysts was <1 hr. The reaction proceeded cleanly and without isocyanate losses. Reaction proceeded similarly in acetonitrile and TBP, although much slower than in DMSO. The best t_{1/2} for acetonitrile and TBP were 6 and 8 hrs., respectively. The best t_{1/2} for DMF and butyl acetate was roughly 10 hrs., although isocyanate losses of>20% were noticed for both solvents. Reaction in methylene chloride proceeded cleanly but very slowly (t_{1/2}>>2 days). The two worst solvents were tetramethyl-1,6-hexanediamine (THDA), and 1-methylpyrrolidin-2-one (MP). No signs of any desirable reaction were found in these two solvents, presumably due to consumption of butyl isocyanate in secondary reactions. Spikes of butyl isocyanate in each of these solvents revealed a complete consumption of the isocyanate by solvent impurities. Although not proven, secondary and/or primary amines present as impurities in these solvents might be the reason for such large isocyanate losses.

Progression of the spontaneous DAN-butyl isocyanate reaction in 5 other solvents (1,4-dioxane, di(ethylene glycol)dibutyl ether, toluene, hexamethylphosphoramide, and pyridine) was very poor compared to DMSO and resembled all other solvents. No sign of the desired reaction was found for di(ethylene glycol)dibutyl ether and hexamethylphosphoramide, similar to THDA and MP. The non-catalyzed reaction in toluene, 1,4dioxane, and pyridine proceeded cleanly, but very slowly, with 10%, 20%, and 40% completion in 10 hrs., respectively. Butyl isocyanate losses of 15% were seen for pyridine.

The catalytic power of various catalysts can be compared by looking at their reaction rates within each solvent. The best catalysts in all solvents but DMSO accelerated reaction of DAN with butyl isocyanate typically no more than 2-3 times. These catalysts had a marginal influence on the rate of DAN and butyl isocyanate reaction and were considered unsatisfactory.

To separate the catalyst from the solvent effect in the case of DMSO, the best combination (DMSO+C₁, C₅, or C₆) was studied in more detail. The reaction kinetics in DMSO at the typical DAN concentration of 5×10⁻³ M was investigated for the non-catalyzed reaction, and for catalysts C₁, C₅, and C₆ at a concentration of 5×10⁻³M, namely 10 times higher. The reaction in all 3 cases proceeded essentially at the same speed, regardless of the catalyst (FIG. 5). These results indicate that DMSO itself is playing a much stronger catalytic role in the DAN-butyl isocyanate reaction than the designated catalysts, and suggest that other catalysts somehow harmed the reaction.

Dibutylin dilaurate at 5×10⁻⁴M in DMSO was also an inefficient catalyst, very similar to C₁, C₅, and C₆. Even at much higher concentrations, these catalysts proved inefficient.

Summary

The derivatization reaction of DAN with two model monoisocyanates-butyl and phenyl isocyanate-proceeded best in DMSO. The catalysts of the isocyanate-alcohol reaction were inefficient in accelerating the DAN-isocyanate reaction. However, the spontaneous reaction of DAN with aromatic isocyanates was almost instantaneous and comparable to that of commercial aliphatic amine reagents with aliphatic isocyanates. The corresponding DAN reaction with butyl isocyanate is slower. The adequacy of this reaction rate for air sampling of aliphatic isocyanates was tested in the field and is described below.

### Example 2

### Finding Optimal Conditions (Solvent, Temperature, and Acid Catalyst) for the Cyclization Reaction to Form Perimidone

Experimental Design for Optimizing the Cyclization Step

DMSO was chosen from Example 1 as the best solvent for derivatization. Finding adequate reaction conditions for fast and efficient cyclization in DMSO would be desirable, as this would minimize the need for further sample processing.

Six acids of variable strength in water and DMSO were tested at two concentrations, 0.1 and 1.0 N (approximately 1.0 and 10% v/v, respectively). The acids were glacial acetic acid (AcOH, 99.99+%), trifluoroacetic acid (TFA, 99+%), benzenesulfonic acid (BSA, 97%), hydrochloric acid (HCl, 37%), sulfuric acid (H₂SO₄, 97%) and Amberlyst® 15 ion-exchange resin with sulfonic acid functionality (4.7 meq/g). The resin offered desirable qualities for field use.

Solutions of the 2 purified model ureas, DANPU and DANBU, in DMSO were prepared at 1.1×10⁻³ M. Acids were directly added into the urea solutions to give a 0.1 and 1.0 N concentration. Aliquots of 100 μL were taken from the reaction vial at appropriate time intervals during a certain period and quenched with 900 μL of 6% triethylamine (TEA) in acetonitrile. Chromatographic conditions were maintained the same as during Example 1. Acetic acid and Amberlyst® (10% v/v) were further tested at 100° C. Acetic acid was also tested at 20, 30, and 50% v/v at ambient temperature and 100° C. Perimidone stability was tested in all acids at 10% v/v, except for Amberlyst®.

Results of Optimizing the Cyclization Step

FIG. 7 shows cyclization progress of DANBU and DANPU in DMSO for 6 acids at 1N and 0.1 N, respectively. Some clear patterns are seen with regard to acid strength and urea class. First, cyclization of both ureas is instantaneous at 1N acid compared to 0.1N for 4 strong acids (TFA, BSA, HCl, and H₂SO₄) in DMSO. Cyclization at 1 N was slow for the weak acid AcOH, and much slower for Amberlyst®. The poor performance of Amberlyst®, despite its strong benzenesulfonic acid functionality, could be due to a slow proton transfer rate from the resin to DMSO. Cyclization of both ureas was so slow at 0.1N acid that other competitive reactions became universally present to some extent for all acids. With regard to side reactions, cyclization of DANPU was very problematic. Acetic acid at 10% was the only acid tested for which cyclization of DANPU was completely clean, although the reaction was slower. Almost in all other cases the side reaction product was the same, despite its unknown structure. Perimidone was found to be very stable under identical acidic conditions. Cyclization yield of DANPU in all acids but AcOH after perimidone concentration reached a stable value was 30-70% in 1N acid, and 20-25% in 0.1N acid. Because the performance of DANPU in these acids was judged unsatisfactory due to substantial side-reactions, no further investigation was conducted on the issue of side reaction products and its mechanism.

In contrast to DANPU, cyclization of DANBU in 1N HCl, TFA, BSA, and H₂SO₄ was very clean, and with 90-100% yield. Even at 0.1N acid, the yield of DANBU cyclization reached near 90%, although after a few hours. Cyclization of DANPU in 0.1N acetic acid was very slow and inefficient, yielding only 30% perimidone after 28 hours. No reaction took place for DANBU under these conditions. Cyclization of DANPU and DANBU in 100% AcOH was completed in <2 minutes and 30 min. (95% in 5 min), respectively.

The above data proved a very complex interplay of DMSO, acid concentration, acid strength and its anion in DMSO, and urea type. It is obvious that a very strong acid is not necessarily a better one for cyclization, and that poor correlation between acid strength and cyclization yield made prediction of the best acid difficult. Therefore, another avenue was considered-speeding up the slow and predictably clean cyclization with AcOH at higher acid concentration and/or temperatures. The cyclization rate of both ureas increases proportionally to the AcOH content and temperature (FIG. 8, for 100° C).

Cyclization was instantaneous for AcOH>30% at 100° C. for both ureas. Cyclization in 50% AcOH progressed fast even at room temperature, reaching completion in about 30 min for DANPU and about 60 min for DANBU. The AcOH content and temperature can be fine-tuned to fit the particular application. Under most conditions, the cyclization step has no time limitations, and 50% AcOH at room temperature is more than sufficient. However, time might be important in field applications. In such a case, 50% AcOH and a gentle warming of the solution up to 40-50° C. will suffice to complete cyclization in a few minutes.

Summary

Cyclization of DANBU and DANPU in DMSO/AcOH mixtures containing 30% AcOH or more proceeds efficiently and quantitatively within a short period of time. Furthermore, cyclization can proceed efficiently in DMSO and no solvent exchange step or other sample preparation is required between steps 1 (Example 1) and 2 (Example 2).

### Example 3

### Investigation of Kinetics and Yield for the DAN Reaction with a Variety of Commercial Isocyanates (Monomeric and Polymeric)

Four monomeric and 4 polymeric products were chosen for further method testing under best conditions from Examples 1 and 2 of model isocyanates (phenyl and butyl isocyanate). Monomeric diisocyanates were 1,6-diisocyanatohexane (1,6-HDI) 98%, tolylene 2,4-diisocyanate (2,4-TDI) 95%, 4,4′-methylenebis(phenyl isocyanate) (4,4′-MDI) 98%, and isophorone diisocyanate (IPDI) 98%. Four prepolymeric products were poly(phenyl isocyanate)-co-formaldehyde) (polymeric MDI) with average molecular weight ca. 375, poly(propylene glycol), tolylene 2,4-diisocyanate terminated (polymeric TDI) with an approximate 8.4% NCO content and average molecular weight of 1,000, N3300 (HDI-isocyanurate), and Z4470 (IPDI-based polyisocyanate). N3300 and Z4470 were supplied by the Bayer Corporation (Pittsburgh, Pa.). The NCO content of four prepolymers, independently determined by titration, was MDI prepolymer 29.8%, TDI prepolymer 6.7%, N3300 21.6%, and Z4470 12.1 %.

The total NCO concentration of these products in the reaction vial was about 1×10⁻⁴N, similar to butyl and phenyl isocyanate. DAN concentration in DMSO was increased to 5×10⁻²M in order to accelerate reaction for aliphatic isocyanates and eliminate potential losses of aromatic isocyanates with water.

### Experimental Design for Reaction Kinetics and Yield of Commercial Isocyanates with DAN

Derivatization and cyclization kinetics were studied only for the four monomers during 30 min., following the same protocols as for the monoisocyanates. An equal amount of acetic acid (50% final volume) was added for cyclization after complete derivatization, and 6% TEA in acetonitrile was used for quenching. Fully MAP-derivatized monomers, potentially formed as a result of quenching during derivatization kinetics, provided additional evidence on the derivatization progress.

The two-step method, without kinetic investigation of both steps, was employed for four prepolymeric products and four monomers. Derivatization was allowed to proceed for at least 5 min. for aromatic products at room temperature, and one hour for aliphatics. Cyclization was allowed to proceed for at least 30 min. at room temperature, although no time restraints existed for this step. The overall two-step method efficiency was determined as perimidone concentration (a measure of total NCO groups found)/total NCO added, both in normality. The total NCO added was calculated from bulk dilution, as total NCO added=NCO % in the product×mass of bulk product/volumetric dilution factor. NCO % for prepolymers was determined by titration. In addition, the total NCO added was independently determined from derivatization with MAP as follows. For monomeric diisocyanates, total NCO(N)=UV370 area of isocyanate-MAP urea/response factor of MAPHDL whereas the polyisocyanates total NCO(N)=UV370 total area of all MAP-isocyanate peaks/response factor of MAPHDI. Cyclization in the above reactions took place in the presence of excess DAN. The chromatography for the cyclization kinetic study of monomeric diisocyanates was maintained essentially the same as previously described for monoisocyanates, except that it was prolonged by 3 min. at the 95% acetonitrile/5% pH 6.0 buffer to elute the heavier MAP derivatives. For all other experiments where the only concern was perimidone quantification, its separation was achieved with an isocratic mobile phase 23% acetonitrile/77% pH 1.6 buffer for 10 min. at the same flow rate of 1.5 mL/min (FIG. 5B). The highly acidic pH accelerates selectively all basic compounds (excess DAN, and reaction byproducts primary amines), without having any effect on the neutral perimidone.

Experimental Results for Reaction Kinetics and Yield of Commercial Isocyanates with DAN

Derivatization of monomeric diisocyanates (FIG. 9A) followed a very similar pattern to that of representative monoisocyanates. The reaction of two aromatic monomers, 2,4-TDI and 4,4′-MDI with 5×10⁻² M DAN in DMSO was instantaneous and could not be measured. The yield was essentially 100% for both aromatic monomers. Derivatization of aliphatic monomers, 1,6-HDI and IPDI, proceeded much more slowly, reaching completion in about 45 min for both aliphatic monomers. However, the non-reacted isocyanate with both NCO groups intact, determined from MAPHDI or MAPIPDI derivative, dropped to 50, 20 and 5% after 2, 5, and 13 min, respectively. Similarly, free IPDI dropped to 56, 19 and 2% in 2, 5, and 13 min., respectively.

The cyclization kinetics of fully derivatized DAN ureas of monomeric diisocyanates are shown in FIG. 9B. The cyclization rate of DAN ureas at 25° C. and 50% AcOH followed this order. 4,4′-MDI>2,4-TDI>1,6-HDI>IPDI. However, differences in the cyclization rate were larger between the class of aromatic and aliphatic isocyanates, and smaller within each class. Cyclization was completed in approximately 30 min for 4,4′-MDI and 2,4-TDI and an hour for 1,6-HDI and IPDI.

The derivatization and cyclization kinetic data for monomers predict a relatively problem-free derivatization and cyclization of prepolymers.

Unfortunately, kinetic data for prepolymers on the individual steps could not be obtained. The only measure of success for prepolymers is the two-step efficiency, i.e., perimidone obtained/isocyanate added. These results are given in Table 1 for monomers and prepolymers.

TABLE 1
Two-step method efficiency for monomers and prepolymers.

| | | NCO concentration (×10⁻⁴N) determined by each method | | | | | |
|---|---|---|---|---|---|---|---|
| | | | | Perimidone | | | Method efficiency^{B} |
| Isocyanate type | Name | Bulk dilution^{A} | MAP derivatization | First | Second | Third | mean (range) |
| Monomer or monoisocyanate | 2,4-TDI | 1.05 | 1.01 | 1.02 | 0.94 | — | 0.97 (0.96-0.98) |
| | 4,4′-MDI | 1.03 | 1.04 | 1.03 | 0.99 | — | 0.95 (0.91-0.99) |
| | Phenyl isocyanate | 1.09 | 0.91 | 1.13 | 0.96 | 1.10 | 0.98 (0.88-1.04) |
| | 1,6-HDI | 1.01 | 0.98 | 0.84 | 0.75 | 1.04 | 0.87 (0.74-1.03) |
| | IPDI | 1.03 | 0.93 | 0.92 | 0.71 | 0.95 | 0.83 (0.69-0.92) |
| | Butyl isocyanate | 1.23 | 1.05 | 1.07 | 1.0 | 1.41 | 0.94 (0.81-1.15) |
| Prepolymer | 2,4-TDI based | 1.22 | NA | 1.27 | 1.27 | — | 1.04 (1.04) |
| | 4,4′-MDI based | 1.24 | NA | 1.17 | 1.14 | — | 0.93 (0.92-0.94) |
| | N3300 (HDI) | 1.84 | NA | 0.60 | 0.68 | — | 0.35 (0.33-0.37)^{C} |
| | Z4470 (IPDI) | 1.29 | NA | 0.39 | 0.45 | — | 0.32 (0.30-0.35)^{C} |

^{A}NCO content for prepolymers was determined by titration and was 7.1% for TDI prepolymer, 30.3% for MDI prepolymer, 21.6% for N3300, and 12.1% for Z4470.
^{B}Method efficiency = NCO concentration from perimidone/NCO from bulk dilution. Numbers are the average of two independent bulk dilutions.
^{C}Efficiency reached a maximum of 40% for both prepolymers when derivatization time was prolonged from 1 to 2 hours, being the same as for overnight derivatization.

There is a clear distinction in method performance between the aromatic and aliphatic class. For aromatics, whether monoisocyanates, monomers, or prepolymers, the overall yield is in the high 90s. The DAN method performance for aromatic isocyanates is very satisfactory. On the contrary, the overall DAN method yield for aliphatic monomers and butyl isocyanate is approximately 90%, and for the two aliphatic prepolymers, N3300 and Z4470, the yield is only one-third of the expected. The yield improved only slightly for both prepolymers when the derivatization time was increased to 2 hours, being the same as for overnight. In order to provide definitive evidence that derivatization step was not the limiting step, an exactly 2-fold excess of DAN was allowed to react with Z4470 (0.5M) in DMSO at room temperature. The excess DAN was quantified one hour later and the next day. In both cases, the excess amount of DAN was exactly half of the original value. This experiment strongly suggests that derivatization is completed within the expected time frame and the problem with aliphatic prepolymers more likely relates to inefficient cyclization. Acetic acid (50% final volume v/v) was added in the above-derivatized solutions one day later. The yield was about 24% for both solutions. It is unclear why aliphatic prepolymeric isocyanates would undergo cyclization much more inefficiently than their respective monomers.

Summary

The derivatization and cyclization kinetics of diisocyanates followed closely that of their respective model monoisocyanates, phenyl and butyl isocyanate. The overall two-step yield of the DAN reaction with aromatic isocyanates to give perimidone was very high, regardless of the isocyanate type-monoisocyanate, diisocyanate, or polyisocyanate. The DAN-based bifunctional nucleophilic scheme is considered successful for the whole class of aromatic isocyanates. The scheme also worked satisfactorily with aliphatic monoisocyanates and diisocyanates, but not with aliphatic polyisocyanates, because they appear to undergo an inefficient cyclization under conditions tested.

### Example 4

### Investigation of Separation of the Cyclization Product (Perimidone) from Excess DAN and Other Reaction Products

HPLC Analysis

Best chromatographic separation of perimidone from other reaction products or excess reagent is achieved with a low pH mobile phase. The low pH mobile phase takes advantage of the fact that perimidone is a neutral compound and its elution time for a particular chromatographic system will depend largely on the organic content of the mobile phase. Excess DAN, reaction by-products (which are primary amines), and potentially other breakdown products of DAN are basic compounds. As such they will respond quickly to a sufficiently acidic mobile phase.

It was found that 20-25% acetonitrile and a pH 1.6 mobile phase accelerate elution of all basic compounds from the analytical column, while allowing sufficient time for perimidone to be separated from them. FIG. 2B illustrates easy separation of perimidone from excess DAN and other reaction byproducts under an acidic mobile phase.

The same principle can be used successfully for sample cleaning in a field application. A cation-exchange solid phase extraction (SPE) seems very attractive. Normal phase separation is also possible.

GC/MS Analysis

DAN and perimidone were analyzed on a HP 6890 gas chromatograph with a HP 5973 mass selective detector. The column used was a 30 m RtX-5 amine column (Restek). The injector temperature was set at 300° C. The mass spectrometer scanned over the range of 100-200 m/z. Several column temperatures and temperature programs were investigated, with 250° C. isothermal found to give good results. At this column temperature, perimidone elutes at 10.3 min, while excess DAN elutes at 4.5 min. It was also determined that the DAN-acetone adduct is easily separated from perimidone by GC. Given the large separation of these compounds, a higher column temperature could be used to give earlier elution and shorter analysis times. Based on the signal-to-noise ratio of the molecular ion signal of the perimidone standard, the LOD is estimated to be about 1 μg/mL when scanning 100-200 m/z. Selected ion monitoring of the molecular ion could be expected to give an additional order of magnitude improvement in detection limit. The mass spectrum of perimidone is dominated by 184 (the molecular ion) and 166 (M-H₂O). Monitoring the ratio of these two ions would ensure that there are no compounds present that interfere with the accurate quantification of the perimidone.

DAN-AcOH Side Reaction

Originally it was observed that blank reagent (DAN in DMSO) when mixed 15 with AcOH gives rise to an interfering peak. Repeated attempts to separate it from perimidone under various chromatographic conditions (pH and organic phase composition) failed. The GC/MS investigation confirmed, by studying the molecular ion of perimidone M⁺ 184, that this interfering peak was perimidone. The rate of this side reaction of perimidone formation was studied.

The rate of perimidone formation was found to be linear (FIG. 10) over one week (perimidone [μg/mL]=0.137×time [hrs], perimidone [M]=7.46×10⁻⁷×time [hrs], R²=0.99), but it is significantly slower than the cyclization rate of DAN ureas. The positive error introduced in the first hour is negligible. This time is sufficient for completion of the primary cyclization reaction even for aliphatic diisocyanates. As one would expect, this reaction is favored by high reagent (DAN, AcOH) concentration, temperature, and time. The unwanted side reaction was successfully stopped by addition of 10 μL acetone in the reaction mixture (about 3 times excess to DAN), which consumes completely and almost instantaneously (confirmed by HPLC) all excess DAN forming 2,2-dimethyl-2,3-dihydoperimidine.

It was found that acetone addition earlier than 20 min cyclization time would reduce the overall yield, presumably as a result of its reaction with the DAN ureas to form N-substituted 2,2-dimethyl-2,3-dihydoperimidines. The perimidone amount in samples remained constant for at least a week later, whereas in blanks no trace of perimidone was found when acetone was added 20-45 min from to of cyclization. These findings suggest that blank reagents should always be run with, and subtracted from, the samples. Because 2,2-dimethyl-2,3-dihydoperimidine is a stronger base than DAN itself, it responds quickly to a strong acidic pH and similar to DAN it does not interfere with perimidone chromatography (FIG. 2B).

Internal Standards

Given the potential volumetric error for this particular application, it can be desirable to use internal standards. Two substances, 1-fluoronaphthalene and 1,8-naphthalimide, were selected as potential candidates based on criteria of chemical inertness, structural similarity to perimidone, and their natural absence in the workplace. 1,8-Naphthalimide was more compatible with the chromatography in use (comparable retention to perimidone), whereas 1-fluoronaphthalene was retained much stronger than perimidone and required an organic gradient. Therefore, 1,8-naphthalimide was preferred as the internal standard. It was proved that 1,8-naphthalimide, when added in the DAN/DMSO solution prior to derivatization, was inert towards NCO groups. The internal standard corrected the perimidone values from the external standard by about 1%.

In addition to studying all issues included in the proposal, some other important work has been completed. This includes investigation of the water effect on derivatization and cyclization, sampling with DAN-impregnated filters, and development of a draft sampling and laboratory analysis method protocol.

Water Effect on Derivatization and Cyclization

High hygroscopicity of DMSO could cause substantial amounts of water to be collected during sampling with an impregnated filter or impinger. Therefore, it is important to know the influence of water on derivatization and cyclization. Water effect on derivatization of butyl and phenyl isocyanate with DAN was studied for a water:DMSO mixture of 30:70% v/v. Butyl isocyanate 1.2×10⁻⁴N was allowed to react with 5×10⁻² M DAN, whereas 1.1×10⁻⁴N phenyl isocyanate was allowed to react with either 5×10⁻³ or 5×10⁻² M DAN. Derivatization kinetics were studied over one hour for both isocyanates, following an identical protocol as previously described for Example 1. Cyclization of pure DANBU and DANPU was tested in a variable DMSO/water mixture containing 10, 30, and 50% water v/v. DANPU and DANBU concentration was 1.0×10⁻³, 1.4×10⁻³, and 1.8×10⁻³ M, for the 50, 30, and 10% water, respectively. Cyclization was catalyzed by addition of an equal volume of glacial acetic acid (500 μL) into (500 μL) urea solutions in the DMSO/water mixture. Cyclization kinetics were studied following the same protocol in Example 2.

Results

Thirty percent water in DMSO, which is a relatively high concentration, resulted in 30% losses for phenyl isocyanate at 5×10⁻³M DAN. No losses were observed at 5×10⁻²M DAN. Reaction was instantaneous in both cases. Since this experiment was a worst case scenario, it can be predicted that losses of aromatic isocyanates in reaction with water can be eliminated by simply increasing DAN concentration above a certain level, in this case 5×10⁻² M.

Surprisingly, water had an accelerating effect on the derivatization rate of butyl isocyanate with DAN in DMSO. Derivatization was completed without losses in less than 7 min for 30% water in DMSO, representing a 10+ times rate increase from pure DMSO. Derivatization at 10% water in DMSO was completed in 15 min or less, confining that the accelerating effect of water on the reaction was real. These results could be explained in light of the strong water-DMSO interactions and hydrogen bond formation.

Water effect on cyclization could not be predicted. However, water concentrations up to 50% clearly did not have any negative impact on cyclization under experimental conditions tested.

Sampling with DAN-Impregnated Filters

The main concern for filter sampling with a slow reacting derivatizing reagent is potential vapor breakthrough. Aromatic isocyanates react very fast with DAN, and it is believed their breakthrough from the filter is very unlikely. Potential vapor losses to competing reactions with water or polyols could be eliminated by using a reagent concentration above a certain level. Because the slow reacting aliphatic isocyanate vapors have the greatest breakthrough potential, HDI monomer vapors were used to test for filter breakthrough.

A human exposure inhalation chamber was used to generate constant HDI monomer vapors. The HDI vapor concentration in the chamber was monitored with an Autostep® direct reading instrument. The vapor concentration in the chamber was conservatively maintained at much higher concentrations than expected in the workplace and varied little (range by Autostep®, 120-152 ppb). The temperature and humidity in the chamber were maintained constant at 25° C. and 50%, respectively. All filters used for the study were 25-mm quartz fiber filters mounted on Delrin cassettes (Gelman Laboratory, Ann Arbor, Mich.). MAP-impregnated 25-mm quartz filters were used side-by-side with all DAN-impregnated filters (via a Y connection) for an independent determination of the HDI vapor concentration.

These filters were impregnated with 500 μg MAP, and at the end of sampling were 25 transferred into jars containing 5 mL of 5×10⁻⁴ M MAP in acetonitrile. The amount of DAN on filters was set at 3 discrete loads: 0.158, 1.58, and 15.8 mg/filter. For each DAN load, some DMSO (0, 20, 200 μL/filter) was added on the filter in hope of improving the derivatization efficiency. Duplicate samples were collected for each combined DAN-DMSO level. MAP-impregnated backup filters, same as the ones used for side-by-side sampling with DAN-filters, were used to measure the amount of HDI monomer breakthrough. Sampling time varied in the range of 7-14 min, with 10 min being the typical duration. The flow rate was 2 L/min. DAN filters were transferred immediately following sampling into 3 mL of 5×10⁻² M DAN in DMSO.

Results

Table 2 gives the percent HDI vapor breakthrough on the front filter (% breakthrough=100×MAPHDI in backup filter/MAPHDI in side-by-side MAP filter).

TABLE 2
Breakthrough^{A, B} (%) of HDI vapors
from DAN-impregnated filters.

| | DMSO (μL/filter) | | |
|---|---|---|---|
| DAN (mg/filter) | 0 | 20 | 200^{C} |
| 0.158 | 52 | — | 36 |
| 1.58 | 2.7 | 7.8 | — |
| 15.8 | 1.1 | 1.0 | 5.3 |

^{A}Breakthrough % = 100 × MAPHDI on backup filter/MAPHDI on side-by-side filter, MAPHDI is the amount of the MAP derivative of HDI
^{B}Each completed cell has at least two pairs of DAN-impregnated filters
^{C}Filters with 200 μL DMSO had a substantial pressure drop and pumps would fail frequently.

Negligible HDI vapor breakthrough was seen at DAN load of 1.58 mg/filter or higher. Contrary to our expectations, DMSO not only did not reduce breakthrough, but instead it facilitated it. It is unclear why the best solvent for DAN derivatization of isocyanates would enhance the breakthrough. It is likely that factors other than reactivity play a role in this process. Nevertheless, the data suggest that DAN-impregnated filters loaded with at least 1.5 mg/filter can be successfully used for sampling of isocyanates.

Draft Sampling and Laboratory Analysis Method Protocol

The developed method uses 1.5 mg DAN on a filter for air sampling. The filter should then be transferred into a DAN solution in DMSO following sampling. Derivatization of aromatic isocyanates with DAN is almost instantaneous and a few minutes would be sufficient for completion of the derivatization step. Then, an equal volume of glacial acetic acid is added to an aliquot of this solution (1 mL) or the whole solution for a final 50% AcOH concentration. Cyclization of aromatic isocyanates at room temperature should be completed in approximately 30 min. A gentle warming of the solution for a few minutes (˜5 min) to 50° C. will suffice to complete cyclization. Ten μL acetone is then added into the solution to consume the excess DAN, not earlier than 20 min if cyclization takes place at room temperature, but not later than an hour. Reagent blanks should also be used with samples. After cyclization is completed, the sample can be analyzed directly with an HPLC mobile phase consisting of 23% acetonitrile/77% phosphate formate buffer pH 1.6 for 10 min at a flow rate of 1.5 mL/min. Perimidone could be detected at the stronger 230 nm band or the more specific 322 nm wavelength or by fluorescence detector. Alternatively, perimidone can be analyzed by GC/MS in the selective ion monitoring mode at M⁺ 184. For a field application of the method, excess reagent can be easily separated either in a normal or reverse phase cartridge.

Summary

Of the 13 solvents tested, which cover a variety of chemical class range and physicochemical properties, DMSO is the only one for which derivatization reaction of DAN with isocyanates proceeds at satisfactory rates. Both reactions of DAN, with butyl isocyanate and phenyl isocyanate, in DMSO were accelerated approximately 15 times compared to acetonitrile, all reaction conditions being the same. Phenyl isocyanate reacted over 3 orders of magnitude faster (1800 times) than butyl isocyanate in DMSO. Additionally, derivatization of four monomeric diisocyanates in DMSO proceeded at comparable speeds to that of monoisocyanates. Most importantly, their derivatization kinetic data indicate that reactivity of the second NCO group in monomeric isocyanates is not adversely affected by derivatization of the first group.

Catalysts generally used in practice for isocyanate-alcohol reactions were found to be ineffective for the present amine (DAN)-isocyanate reactions.

The significantly better performance of DMSO compared to all other solvents suggests that DMSO, beyond providing a reaction medium, is playing an active catalytic role in the reaction. DMSO, given its high dielectric constant and polarity, activates the NCO bond by forming an intermediate complex with it, which is more amenable to a nucleophilic attack by DAN (FIG. 11). For reasons described above, identification of DMSO as a distinctly better reaction medium for the DAN-isocyanate reaction than that of the other solvents is a significant finding.

It was hypothesized that the mechanism of acid-catalyzed cyclization very likely follows an addition-elimination scheme as depicted in FIG. 12. According to this mechanism, the acid-catalyzed cyclization of DAN ureas is very sensitive to the pH of the medium. The reaction rate would be the highest when protonation of C═O is efficient, and yet there is enough free amine groups to ensure a nucleophilic attack on the C⁺ center. Differences in performance of various acids in DMSO have to do with the acidity scale in DMSO. The acidity scale in DMSO changes considerably from that in water. The acid strength in water (pK_{a,w}) and DMSO (pK_{a,DMSO}) for the acids tested as catalysts in cyclization varies as follows: HCl (pK_{a,w}=−8; pK_{a,DMSO}=1.8), TFA (pK_{a,w}=0.5; pK_{a,DMSO}=3.45), BSA (pK_{a,w}=2.6; pK_{a,DMSO}=˜6), H₂SO₄ (pK_{a,w}=strong; pK_{a,DMSO}=˜HCl), and AcOH (pK_{a,w}=4.75; pK_{a,DMSO}=12.3). All acids tested are orders of magnitude weaker in DMSO than in water (ΔpKₐ DMSO-water=3-10). Nevertheless, they all, with the exception of AcOH, remain strong acids in DMSO. The free amine group of DAN ureas (pK_{a,w}=˜4.5—by analogy to 1,8-DAN, pK_{a,w}=4.44; pK_{a,DMSO}=˜4 by analogy to aniline, pK_{a,DMSO}=3.6) will be 99+% protonated in HCl and H₂SO₄, 50+% in TFA, and essentially unprotonated in AcOH. The data, however, indicate a more complex interaction between the acid and DMSO, especially with respect to proton (H+) mobility in DMSO and solvation of intermediate charged species.

Water did not have any effect on cyclization, and if adequate DAN concentrations are maintained, losses of aromatic isocyanates to water or polyols could be eliminated.

Sampling with DAN-impregnated filters is possible for both aromatic and aliphatic isocyanates. The reaction rate of aromatic isocyanates with DAN in DMSO is comparable to that reaction of commercial aliphatic amine reagents with aliphatic isocyanates. In addition, negligible breakthrough of HDI vapors for DAN-impregnated filters with >1.58 mg DAN/filter at ˜150 ppb HDI suggest sampling of aliphatic isocyanates is also possible.

The blank reagent and AcOH undergo a reaction of unknown mechanism to produce perimidone. The rate of this undesirable reaction is significantly slower than the primary cyclization reaction, and the reaction is successfully stopped by consuming excess DAN with acetone. However, it would be more desirable to understand its mechanism and inhibit the reaction completely.

Polymeric aliphatic isocyanates do not perform well, and the recent data indicate that the cyclization step is inefficient. Finding a solution to this problem would be useful, as this will expand the DAN method applicability to all isocyanates.

Since the method is based on laboratory tests, it is desirable to further test the method in a variety of field situations, as well as for potential interferences. A field version of the method would also require some research into sample preparation and the robustness of field analytical equipment.

### Example 5

### Investigation of Separation of the Cyclization Product (Perimidone) from Excess DAN and Other Reaction Products

A method protocol was developed and described above.

Briefly, isocyanates were collected in a solution of 0.05M DAN in DMSO with an impinger or DAN impregnated filter (loaded with ˜1.6 mg DAN/25-mm filter), which would then be transferred in the field immediately post-sampling into a 0.05M DAN solution in DMSO. After derivatization reaction has reached completion (minutes to hours depending on the isocyanate type, concentration, and environmental conditions), an aliquot of this solution is mixed with an equal volume of glacial acetic acid (50% v/v final). Acetic acid catalyzes cyclization of DAN ureas to form perimidone. One hour into cyclization 10 μL of acetone are added to consume excess DAN, which under these conditions undergoes an undesirable secondary reaction to produce perimidone. This reaction is much slower than the primary cyclization reaction and is completely negligible within the first few hours. The sample then undergoes a cleanup solid phase extraction procedure (SPE), which is currently under development, and perimidone is analyzed by GC-MS using selective ion monitoring at m/z 184 (M⁺) and 166 and deuterated d₆-perimidone as the internal standard or by high performance liquid chromatography (HPLC) using ultraviolet detection at 230 and/or 322 nm and external perimidone standards.

We have developed a cleanup protocol. Isocyanates have been spiked into the DAN /DMSO solution. Sampling (collection+derivatization) efficiency has been studied.

Methods

GC/MS Analytical Finish

Initially (as described above), DAN and perimidone were analyzed on a HP 6890 gas chromatograph with a HP 5973 mass selective detector. The GC was equipped with a Merlin Microseal™ high pressure septum and a glass, base deactivated 4 mm ID gooseneck injector liner. Some experiments were conducted with the injector liner containing glass wool and others without the glass wool. The injection mode used was pulsed splitless. The column used was a 30 m RtX-5 amine column (Restek). The injector temperature was set at 300° C. The mass spectrometer scanned over the range of 100-200 m/z. Several column temperatures and temperature programs were investigated, with 250° C isothermal found to give good results. At this column temperature, perimidone elutes at 10.3 min, while excess DAN elutes at 4.5 min. It was also determined that the DAN-acetone adduct is easily separated from perimidone by GC. Given the large separation of these compounds, a higher column temperature could be used to give earlier elution and shorter analysis times. Better performance was observed when there was no glass wool in the injector liner. Based on the signal-to-noise ratio of the molecular ion signal of the perimidone standard, the LOD is estimated to be about 1 μg/mL when scanning 100-200 m/z. Selected ion monitoring of the molecular ion could be expected to give a substantial improvement in detection limit. The mass spectrum of perimidone is dominated by 184 (the molecular ion) and 166 (M-H₂O). Monitoring the ratio of these two ions would ensure that there are no compounds present that interfere with the accurate quantification of the perimidone.

The sensitivity of the analysis under conditions described above was only marginally acceptable and would be unacceptable for small sample sizes. In order to minimize reactivity and be able to use higher temperatures to maximize vaporization of the perimidone, a few modifications were made to the GC inlet system. The Merlin Microseal™ high pressure septum was replaced by a high temperature septum (max 400° C.). The gooseneck injector liner was replaced by a quartz 2 mm ID open ended deactivated liner and high temperature O-rings (max 400° C.) were installed. The inlet was operated in splitless mode rather than pulsed splitless mode, and the injector was set at 375° C. A short, 15 m RtX-5 amine column, 0.5 μm film, 0.25 mm ID was used. The performance under these conditions was dramatically improved with nearly two orders of magnitude increased sensitivity. Selected ion monitoring (SIM) mode was used for mass spectrometry collection of the 184 ion and 166 ion, using a dwell time of 50 ms for each ion. The LOD is estimated to be 5 ng/mL under these conditions. This sensitivity is comparable to that of NIOSH 5525 (MAP), but with greatly improved selectivity.

Method Bias and Interferences

Method Bias

Method bias was determined for six aromatic isocyanate products of which five from Bayer Co. (Pittsburgh, Pa.) were of commercial importance and one prepolymeric product from Aldrich (Milwaukee, Wis.) was included for structural diversity. These products were: Bayer's Mondur® TD 65/35 [65%/35% 2,4- and 2,6-toluene diisocyanate TDI], Mondur® ML [55%/45% 4,4′-/2,4′-methylene diphenyl diisocyanate MDI], Mondur® MR [polymeric MDI], Desmodur® CB 75N (2,4-TDI-trimethylolpropane diluted with 25% ethyl acetate) and Desmodur® IL (2,4-TDI-isocyanurate diluted with 50% n-butyl acetate), and the Aldrich poly(1,4-butanediol), tolylene 2,4-diisocyanate terminated here abbreviates as PBD-TDI. Diluted solutions of isocyanate bulks in methylene chloride were spiked into a 1 mL solution of 0.05M 1,8-DAN in DMSO to give a final concentration of ˜1×10⁻⁴N of the isocyanate group (NCO). The derivatization reaction was allowed to complete for at least 30 min. Then, an equal amount of glacial acetic acid, serving as the catalyst for the cyclization reaction, was added to the derivatizing solution to give a 50% AcOH v/v concentration. The cyclization reaction was allowed to complete for 1 hr at which time 10 μL of acetone were added to the reaction mixture to consume excess DAN. Samples were then analyzed for perimidone by high performance liquid chromatography (HPLC) on a Agilent HP 1100 series instrument. The analytical column was a 5 μm C8 Inertsil 150×4.6 mm column from Phenomenex (Torrance, Calif.). Detailed chromatographic conditions are provided in the above Examples. Each product was diluted, derivatized and analyzed in independent triplicates. Perimidone external standards in the range of 1×10⁻⁶ to 1×10⁻³M were used for quantification.

Simultaneously with 1,8-DAN derivatization, the same amount of isocyanate was spiked into a 1 mL of 5×10⁻⁴M 1-(9-anthracenylmethyl)piperazine (MAP) in acetonitrile and the derivatized MAP isocyanate ureas were analyzed by HPLC using the NIOSH MAP method 5525. Hexamethylene diisocyanate (HDI)—MAP urea was used as external standard for quantification of isocyanates. The chromatographic system was the same for both perimidone and MAP-urea analysis, but chromatographic conditions varied.

The NCO content of each bulk was obtained from Bayer Co. and verified by independent titration using a published protocol.

Interferences

Compounds likely to be found in the workplace that would react with 1,8-DAN to produce perimidone or another interfering analyte, or simply consume the reagent without undergoing cyclization were investigated as likely interferences.

Model compounds, which served as representatives of their respective chemical classes, included: phenyl chloroformate (chloroformates), 2,4-TDI and 1,6-HDI methyl carbamate (aromatic and aliphatic carbamate, respectively), polypropylene glycol PPG (polyester polyols), poly(2-methyl-1,3-propylene adipate) PPA (polyester polyols), acetone (ketone), butyl acetate (esther), air, and CO₂. All chemicals, except 2,4-TDI and 1,6-HDI methyl carbamate and the CO₂ tank, of the highest purity grade available were purchased from Aldrich (Milwaukee, Wis.), and used without further purification. The carbamates were synthesized in the lab from the reaction of respective isocyanates with excess methanol, the solvent was later evaporated and carbamates dried under a gentle stream of N₂.

Two model carbamates were chosen to judge reactivity (of both NH₂ groups) of DAN towards newly formed ureas. All others were chosen to investigate potential DAN consumption from solvents or other paint ingredients, O₂ and CO₂ in air during air sampling and/or subsequent perimidone formation from the reaction of a carbonyl group during cyclization.

Interferences were studied during the two steps of the analytical scheme, the first being the derivatization step under neutral condition, and the second one being cyclization under 50% AcOH. Potential reaction of DAN with any of these compounds was investigated by means of HPLC kinetic studies of the DAN with these interferent compounds over time. All reagents, except phenyl chloroformate and air, were allowed to react with 1,8-DAN in DMSO at concentrations of 0.001 M DAN and 0.01M interferent. DAN consumption, if any, was measured over a period of 24 hrs. Phenyl chloroformate at 0.001M was originally allowed to react with 0.002 M DAN in DMSO. DAN oxidation from O₂ in air was investigated by drawing air through 15 mL 0.05 M DAN in DMSO at 1 L/min for >12 hrs. Interference from CO₂ was studied in a similar design, except air was enriched with technical grade CO₂ to ≦0.6% (as measured by the TSI×direct reading instrument) and drawn through an impinger solution for ˜21 hrs. Aliquots of these solutions were then mixed with an equal volume of AcOH and analyzed for perimidone.

Various ketones were tested relative to acetone for destruction/removal of excess DAN. Results are shown in FIG. 15.

TABLE 4
Performance of ketones for destruction of excess DAN.

| | | perimidone area | |
|---|---|---|---|
| | Time (min) | @UV322 nm | % DANPU Losses |
| DANPU-K1-t₀ | 1 | 1111 | 13.61 |
| DANPU-K1-t₁ | 11 | 1178 | 8.40 |
| DANPU-K1-t₂ | 20 | 1217 | 5.37 |
| DANPU-K1-t₃ | 30 | 1287 | −0.08 |
| DANPU-K5-t₀ | 1 | 1165 | 9.41 |
| DANPU-K5-t₁ | 11 | 1157 | 10.03 |
| DANPU-K5-t₂ | 22 | 1198 | 6.84 |
| DANPU-K5-t₃ | 32 | 1261 | 1.94 |
| DANPU-K7-t₀ | 1 | 1134 | 11.82 |
| DANPU-K7-t₁ | 11 | 1243 | 3.34 |
| DANPU-K7-t₂ | 20 | 1288 | −0.16 |
| DANPU-K7-t₃ | 30 | 1314 | −2.18 |
| DANPU-K8-t₀ | 3 | 1287 | −0.08 |
| DANPU-K8-t₁ | 10 | 1278 | 0.62 |
| DANPU-K8-t₂ | 20 | 1188 | 7.62 |
| DANPU-K8-t₃ | 30 | 1284 | 0.16 |
| DANPU-REF | | 1286 | |
| PRM1E-4M | | 287.4 | |
| PRM1E-3M | | 2827 | |

As can be seen from the data, 2-decanone reacts faster than 3-decanone with DAN. FIG. 16 shows DAN consumption with 2-/3-decanone in Step 2 of a method of the invention.

Cyclization Kinetic for all Tested Isocyanates

Cyclization kinetic was studied for all six model isocyanates mentioned in the bias section over a period of 1 hr.

Results

GC-MS Analytical Finish

Successful separation of perimidone from excess DAN.
Acetone and methanol preferred solvents for MS analysis.
Selective ion monitoring at m/z=184 (M⁺) and 166, and m/z 184/166 ration for purity and/or identity confirmation.
Deuterated perimidone ideal internal standard, but not commercially available.
Estimated instrument LOD 5.5 ng/mL, comparable to UV detection. Linearity range of 2-500 ng/mL.
Overall, GC-MS analysis of perimidone is considered successful.
   Method Bias

Results of the bias experiments are provided in Table 3.

TABLE 3
Method bias for DAN and MAP method.

| | | | Analytical method based on this derivatizing reagent | | | | | |
|---|---|---|---|---|---|---|---|---|
| | | | 1,8-DAN | | | | | |
| Common experimental conditions for | | | Mean | | | MAP | | |
| both reagents | | | perimidone | | | Mean NCO | | |
| | | NCO group | recovery | | | recovery | | |
| | % NCO | (N) spiked | (n = 3 | | | (n = 3 | | |
| Model | in | in reaction | independent | Individual | Mean CV ³ | independent | Individual | Mean CV ³ |
| isocyanate | bulk ¹ | vial ² | experiments) | recoveries | % (n = 3) | experiments) | recoveries | % (n = 3) |
| Mondur ® | 48.2 | 1.2E−04 | 0.97 | 0.92; 1.0; 1.0 | 0.043 | 0.98 | 0.96; 0.96; 1.01 | 0.031 |
| TD 65/35 | | | | | | | | |
| Desmodur ® | 8.3 | 1.2E−04 | 0.90 | 0.88; 0.90; 0.91 | 0.002 | 0.45 | 0.42; 0.44; 0.50 | 0.093 |
| IL | | | | | | | | |
| Desmodur ® | 13.7 | 1.1E−04 | 1.03 | 0.96; 1.05; 1.07 | 0.059 | 0.72 | 0.66; 0.73; 0.78 | 0.082 |
| CB 75N | | | | | | | | |
| Mondur ® | 33.5 | 1.1E−04 | 1.00 | 0.95; 0.96; 1.08 | 0.073 | 1.05 | 0.97; 1.08; 1.08 | 0.062 |
| ML | | | | | | | | |
| Mondur ® | 31.4 | 1.1E−04 | 0.96 | 0.95; 0.96; 0.97 | 0.011 | 0.84 | 0.81; 0.82; 0.89 | 0.054 |
| MR | | | | | | | | |
| PBD-TDI | 8.8 | 1.2E−04 | 0.99 | 0.93; 1.02; 1.03 | 0.056 | 1.12 | 1.10; 1.12; 1.13 | 0.011 |

| | | | | |
|---|---|---|---|---|
| Mean | 0.98 (0.88-1.08) | 0.41 (0.002-0.073) | 0.86 (0.42-1.13) | 0.056 (0.01-0.093) |
| (range) | | | | |

¹ Determined by the titration assay
² This concentration, calculated from bulk dilution factors and % NCO in bulk, was used as reference concentration in determining the recovery.
³ Coefficient of variation or relative standard deviation (RSD) calculated as standard deviation/mean.

Recovery was calculated as follows:

Recovery=(Spiked NCO amount based on bulk dilution−found perimidone)/Spiked NCO amount

Amount of isocyanate spiked and dilution factors were used to calculate the reference NCO value. Quantitative (overall mean recovery=97.4%, range=88-108%) and very reproducible recoveries (overall mean relative standard deviation RSD=4.1%, range of means=0.2-7.3%) for all tested isocyanates indicate that the DAN method is accurate in measuring the total NCO content of a sample derived from an aromatic isocyanate.

The DAN method outperforms noticeably the more standard analytical scheme for isocyanate determination based on individual identification and quantification of MAP-derivatized species in overall accuracy, reproducibility, and simplicity. Because the DAN method measures a single analyte, it is conceivable that the method is simpler and more reproducible. The MAP method showed higher variability (overall mean RSD=5.6%, range of means=0.1-9.3%) in results in comparison to the DAN method. More noticeable is the substantially higher bias of the MAP method for some polymeric aromatic isocyanates. For TDI-isocyanurate the MAP method gave only 45% of the expected NCO content, compared to 90% for DAN. The substantial bias of conventional isocyanate methods, including MAP, towards aromatic prepolymeric isocyanates is well known, although the true reason for such bias is not.

The DAN method shows no significant bias for the analysis of aromatic isocyanate products. This is in contrast to the MAP method which shows considerable bias with some aromatic products.

Interferences

None of the tested interferences appear to react with DAN under neutral conditions, and none gave perimidone during the cyclization step under the conditions of the above protocol. Phenyl chloroformate does react with 1,8-DAN, but the rate of this reaction is very slow. For example at the concentrations tested (0.001M Phenyl chloroformate+0.002M DAN in DMSO) the reaction was immeasurable during the first 24 hrs. In order to verify that a slow reaction was taking place, the experiment was repeated at substantially higher concentrations of phenyl chloroformate (0.02M+0.001M DAN), and repeated assisted from a tertiary amine catalyst (0.02M phenyl chloroformate+0.001M DAN+0.036M triethyl amine). Results in FIG. 13 suggest that phenyl chloroformate is very unlikely to present a problem for isocyanate analysis, because it is not expected to be found at such high concentrations in the workplace.

The carbamates, a polyether polyol, a polyester polyol, and butyl acetate did not react with DAN.

Neither CO₂, nor O₂ present any problem for air sampling with DAN/DMSO solutions, although substantial amounts of water are collected during sampling (˜9% in our experiments). Oxygen was not found to consume DAN during sampling, and CO₂ was not found to yield perimidone after sample work up. Similarly, no problems were identified during air sampling on DAN-impregnated filters for 8+ hrs at 2 L/min in a previous experiment.

Although acetone does not react with DAN under neutral conditions, it was found that acetone interferes with cyclization of DAN urea intermediates if present when acetic acid is added. Acetone is known to react with DAN under acidic conditions. This reaction does not give rise to perimidone and does not interfere with the collection and derivatization efficiency of isocyanates. It does, however, reduce the overall recovery of the isocyanate group as perimidone as a result of acetone ability to react with DAN isocyanate ureas in the presence of AcOH. In our experiments we have found a loss of as much as 20% perimidone. This is a concern because acetone and methyl ethyl ketone are used as co-solvents for isocyanates. The role of the ketone chemical structure in the reaction rate with DAN isocyanate ureas was investigated.

Cyclization Rate

FIG. 14 provides cyclization kinetics data for all six products. Cyclization rate of DAN derivatives of aromatic isocyanates was found to be dependent on the isocyanate structure. Mondur® ML and MR undergo cyclization noticeably faster than the rest of prepolymeric products. Nonetheless, cyclization was completed in 1 hr for all isocyanates under normal conditions and time was not a limiting factor.

Throughout this application, various publications are referenced. The disclosures of these publications in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method for detecting and/or measuring total reactive isocyanate in a sample comprising
a) contacting a bifunctional nucleophilic isocyanate derivatizing agent with a sample, containing or suspected of containing isocyanate, under conditions suitable for the formation of a reaction product capable of detection, and
b) detecting the presence or absence of the reaction product as an indication of the presence or absence of isocyanate in the sample.

2. The method of claim 1 wherein the bifunctional nucleophilic isocyanate derivatizing agent comprises a fused aromatic ring.

3. The method of claim 2 wherein the fused aromatic ring is naphthalene, anthracene, or derivative of naphthalene or anthracene.

4. The method of claim 3 wherein the derivative comprises an alkyl substituent on a ring.

5. The method of claim 4 wherein the alkyl substituent is methyl.

6. The method of claim 1 wherein the two functionalities of the bifunctional nucleophilic isocyanate derivatizing agent are aminos.

7. The method of claim 1 wherein the bifunctional nucleophilic isocyanate derivatizing agent is a naphthalene or a derivative thereof with the two isocyanate derivatizing functionalities attached.

8. The method of claim 1 wherein the bifunctional nucleophilic isocyanate derivatizing agent has the two functionalities on the same plane as the molecular backbone.

9. The method of claim 8 wherein the bifunctional nucleophilic isocyanate derivatizing agent has the two functionalities in the 1 and 8 positions of the naphthalene rings, the 1 and 9 positions of an anthracene, or the equivalent positions for other fused aromatic ring compounds.

10. The method of claim 1 wherein the two functionalities of the bifunctional nucleophilic isocyanate derivatizing agent are capable of forming a 6-membered ring after binding with an isocyanate group.

11. The method of claim 1 wherein the bifunctional nucleophilic isocyanate derivatizing agent
a) has the two functionalities on the same plane as the molecular backbone, and
b) the two functionalities of the bifunctional nucleophilic isocyanate derivatizing agent are capable of forming a 6-membered ring after binding with an isocyanate group.

12. The method of claim 1 wherein the bifunctional nucleophilic isocyanate derivatizing agent has a conformation which forces the two functionalities to have a relative geometry conducive to formation of a cyclic structure after reacting with isocyanate.

13. The method of claim 1 wherein the bifunctional nucleophilic isocyanate derivatizing agent is 1,8-diaminonaphthalene (DAN).

14. The method of claim 1 wherein the structure of the reaction product is independent of that of the isocyanate group thereby forming a single analyte for detection.

15. The method of claim 14 wherein the analyte is quantified as an indication of the quantity of isocyanate in the sample.

16. The method of claim 1 wherein step a) comprises the steps
i) derivatizing the isocyanate with the derivatizing agent in the presence of an effective derivatizing catalyst to form an intermediate and
ii) cyclizing the intermediate in the presence of an effective cyclizing catalyst to form the reaction product.

17. The method of claim 1 wherein the reaction product is a cyclic urea.

18. The method of claim 16 wherein the intermediate is a urea.

19. The method of claim 13 wherein the reaction product is perimidone.

20. The method of claim 1 wherein the presence or absence of reaction product in the sample is detected using chromatographic methods.

21. The method of claim 16 wherein the effective derivatizing catalyst is a solvent.

22. The method of claim 21 wherein the solvent is DMSO.

23. The method of claim 16 wherein the effective cyclizing catalyst is an acid.

24. The method of claim 23 wherein the acid is acetic acid.

25. The method of claim 1 further comprising eliminating excess derivatizing reagent.

26. The method of claim 25 wherein the eliminating excess derivatizing reagent comprises adding acetone prior to step b).

27. The method of claim 20 wherein the chromatographic method is GC/MS.

28. The method of claim 27 wherein the GC/MS analysis comprises use of a high temperature septum, a quartz open ended deactivated injector liner, and high temperature O-rings in the GC inlet; the inlet is operated in splitless mode; and injection is at a higher temperature relative to conventional analysis of the product.

29. A method for detecting and/or measuring total isocyanate in a sample comprising
a) contacting a bifunctional nucleophilic, fused aromatic ring isocyanate derivatizing agent, wherein the two functionalities are amino functionalities in a symmetrical, planar relation to the molecular backbone so as to be capable of forming a cyclic reaction product and capable of reacting with an isocyanate group to form a urea, with a sample under conditions suitable for the formation of the cyclic reaction product capable of detection
wherein the cyclic reaction product's structure is independent of that of the isocyanate group, and
(b) detecting the presence or absence of the cyclic reaction product as an indication of the presence or absence of isocyanate in the sample.

30. A method for detecting and/or measuring total isocyanate in a sample comprising
a) contacting 1,8-diaminonaphthalene with a sample containing isocyanate groups under conditions suitable for the formation of a reaction product capable of detection, and
b) detecting the presence or absence of the reaction product as an indication of the presence or absence of isocyanate in the sample.

31. A method for determining the species of isocyanate in a sample comprising
a) contacting a bifunctional nucleophilic isocyanate derivatizing agent with a sample, containing or suspected of containing isocyanate, under conditions suitable for the formation of an intermediate capable of detection, and
b) detecting the presence or absence of the intermediate as an indication of the presence or absence of isocyanate species in the sample

32. A method for determining the total amount of isocyanate in a sample comprising:
a) contacting the sample with a bifunctional nucleophilic isocyanate derivatizing agent in the presence of an effective derivatization catalyst to form a urea;
b) cyclizing the urea to form a cyclic reaction product using an effective cyclizing catalyst;
c) eliminating unreacted derivatizing agent; and
d) quantifying the amount of reaction product produced.

33. The method of claim 32 wherein the bifunctional nucleophilic isocyanate derivatizing agent is DAN.

34. The method of claim 32 wherein eliminating unreacted derivatizing agent comprises adding acetone.

35. The method of claim 32 wherein the effective derivatizing catalyst is a solvent and wherein the solvent is DMSO.

36. The method of claim 32 wherein the reaction product is quantified using chromatographic methods.

37. A method for determining the amount of individual isocyanates in a sample comprising:
a) contacting the sample with a bifunctional nucleophilic isocyanate-derivatizing agent to form a mixture of ureas;
b) detecting the individual ureas within the sample; and
c) quantifying the amount of urea, wherein the amount of urea corresponds to the individual isocyanate being determined.

38. The method of claim 37 wherein the bifunctional nucleophilic isocyanate derivatizing agent is DAN.

39. The method of claim 37 wherein the urea is quantified using chromatographic methods.

40. A method for determining the total amount of isocyanate on a solid or particle surface comprising
a) contacting a solid or particle surface with a bifunctional isocyanate derivatizing agent;
b) treating the solid or particle surface with acetic acid to form a cyclic reaction product; and
c) quantifying the amount of cyclic reaction product produced.

41. The method of claim 40 wherein the isocyanate derivatizing agent is DAN.

42. The method of claim 40 wherein the solid or particle is polyurethane or dust from wood composites.

43. A kit for detecting and/or measuring total isocyanate in a sample comprising
a) a bifunctional nucleophilic derivatizing agent.

44. The kit of claim 43 wherein the bifunctional nucleophilic derivatizing agent is DAN.

45. A filter for collecting a sample for detecting and/or measuring total isocyanate in a sample comprising
a) an air sample collection filter, and
b) a bifunctional nucleophilic derivatizing agent.

46. The filter of claim 45 wherein the bifunctional nucleophilic derivatizing agent is DAN.

47. A kit for solid phase extraction (SPE) for detecting and/or measuring total isocyanate in a sample comprising
a) a SPE cartridge, and
b) a bifunctional nucleophilic derivatizing agent.

48. The kit of claim 47 wherein the bifunctional nucleophilic derivatizing agent is DAN.
